(19) United States
(12) Patent Application Publication
(10) Pub. No.: US 2026/0297637 A1
(43) Pub. Date: Oct. 1, 2026

(54) FATTY ACID ESTERIFICATION PRETREATMENT PROCESS

(71) Applicant: Novozymes A/S, Bagsvaerd (DK)

(72) Inventors: Rasmus Boeg ALSTRUP, Frederiksberg (DK); Danilo MARQUES DE LIMA, Campinas (BR); Florivaldo Alberto GALINA JÚNIOR, Ribeirão Preto (BR); Lucas KLETTENHOFER, Jundiai (BR); Fernanda RIBEIRO DE FARIA, Araraquara (BR); Graziela Pinheiro BRUNI, Campinas (BR)

(73) Assignee: Novozymes A/S, Bagsvaerd (DK)

(21) Appl. No.: 19/479,248

(22) PCT Filed: Apr. 25, 2024
(86) PCT No.: PCT/EP2024/061347

§ 371 Date: Oct. 27, 2025

(30) Foreign Application Priority Data
Apr. 27, 2023 (EP) 23170320.8
Nov. 15, 2023 (EP) 23210126.1

(51) Int. Cl. C12P 7/649

(52) U.S. Cl. CPC C12P 7/649

(57) ABSTRACT

A process for enzymatic esterification of free fatty acids. In particular, the present invention relates to a pretreatment process for conversion of free fatty acids in oils and fats as feedstock for base-catalyzed transesterification to produce biodiesel.

### FIELD OF THE INVENTION

The present invention relates to a process for enzymatic esterification of free fatty acids. In particular, the present invention relates to a pretreatment process for enzymatic conversion of free fatty acids in oils and fats as feedstock for base-catalyzed transesterification to produce biodiesel.

### BACKGROUND OF THE INVENTION

Most biodiesel is produced from oils and fats through transesterification of glycerides with methanol by use of a strong base catalyst. Glycerides are mono-di- and tri-esters of glycerol and fatty acids. The transesterification reaction will yield methyl-esters by combining the fatty acids of the glycerides with methanol, and these methyl-esters are broadly called Fatty Acid Methyl Esters (FAME). The FAME product of sufficient quality is finished biodiesel. A higher-tier glyceride such as tri-acyl-glycerol (TAG) will become a lower-tier glyceride in the process, such as di-acyl-glycerol (DAG) and by further reaction mono-acyl-glycerol (MAG). In the end, free glycerol is liberated in the process and will precipitate as a polar, liquid heavy phase, which is typically and broadly called crude glycerin. The crude glycerin is further refined to bring (crude) glycerin of higher qualities.

The strong base catalyst is typically deprotonated methanol, namely sodium-methylate, but can be other strong bases. A transesterification reaction does not involve water. It is a direct substitution of an alcohol in an ester bond with a different alcohol. In the case of biodiesel, it is substitution of one glycerol-hydroxyl group with the hydroxyl group on methanol. However, if water is present, even in small amounts, then water can practically act as alcohol in the reaction instead of methanol. This will still yield liberated glycerol in the end but will yield free fatty acids (FFA) rather than methyl-ester biodiesel. This reaction involving water is called hydrolysis—the opposite of esterification.

Most oils and fats comprise a significant amount of FFA after extraction or isolation of the oil from the oilseed or other original material. For example, extracted crude oils such as soybean oil typically comprises FFA in the range of 0.5 to 3 wt % FFA, depending on seasonality and other factors. Other relevant feedstocks comprise significantly higher amounts of FFA, such as used cooking oil, where e.g., 10 wt % FFA can be observed. And FFA stemming from the stripping processes in oil refineries, typically named deodorization, can be found in concentrations exceeding 80 wt % as the fatty acid distillate (FAD) side stream from the refining process. Most of the remainder of all these examples is some mixture of the three main glycerides MAG, DAG and TAG, which are readily converted by the strong base catalyst. As a result, to convert feedstocks comprising FFA using base-catalyzed transesterification, a pretreatment process is warranted, and the typical target FFA level of such pretreatment is FFA<0.1 wt % in the feedstock stream entering the main transesterification reaction. Higher levels of FFA are not prohibitive but come at the expense of catalyst dosage cost, which can be acceptable.

EP4071226 discloses a process for producing fatty acid alkyl esters by catalytic transesterification of triglycerides from an oil and/or fat based crude feedstock containing triglycerides and free fatty acids.

WO2012098114 discloses a method for producing fatty acid alkyl esters, wherein a solution comprising triglyceride, alcohol, water, and glycerol is contacted with a lipolytic enzyme.

WO2021074201 discloses a process for enzymatic esterification of free fatty acids. In particular the invention relates to this process using a flash operation for water removal from enzyme reaction mixture.

There are a range of pretreatment options available to the producer of biodiesel using the base-catalyzed transesterification pathway. Each option has drawbacks and limitations as well as benefits. The present invention provides the significantly improved reaction regarding FFA reduction using an enzymatic pretreatment without a requirement for significant glyceride conversion.

### SUMMARY OF THE INVENTION

The present invention relates to a process for reduction of free fatty acid concentrations in a fatty acid feedstock by enzymatic esterification with alcohol thereby reducing free fatty acid concentrations to levels compatible with a base-catalyzed biodiesel production process, or as a crude oil product suitable as fuel for ships or as a feedstock oil for hydrotreated vegetable oil (HVO) production, comprising steps of:
(a) providing fatty acid feedstock comprising more than 1 wt % Free Fatty Acids (FFA);
(b) contacting said fatty acid feedstock with a composition comprising an esterase and at least one component selected from the group consisting of alcohol, alcohol and glycerol, alcohol and water, alcohol and water and glycerol to form a mixture; and
(c) incubating the mixture of step (b) under conditions allowing the esterase to catalyze esterification of FFA until FFA levels are reduced either:
   i) below 10 wt % if a neutralization step follows the enzymatic esterification reaction;
   ii) below 2 wt % if a base-catalyzed transesterification follows the enzymatic esterification reaction without further FFA reduction; or
   iii) below 10 wt %, below 5 wt %, such as below 2 wt % if neither neutralization nor base catalyzed transesterification follows the esterification step c).

In a further aspect, the present invention relates to a process for producing a crude biodiesel mixture, said process comprising an enzymatic pretreatment according to claim 1, and further comprising at least one separation step of a light and a heavy phase, and comprising at least a base catalyzed transesterification step to produce the crude biodiesel mixture comprising alkyl ester, e.g., FAME.

In a still further aspect, the present invention relates to an enzymatic process for producing an oil suitable as fuel for ships and/or as an oil feedstock for further downstream processing not involving transesterification, comprising the steps of:
(a) providing fatty acid feedstock comprising more than 1 wt % Free Fatty Acids (FFA);
(b) contacting said fatty acid feedstock with a composition comprising an esterase and at least one component selected from the group consisting of alcohol, alcohol and glycerol, alcohol and water, alcohol and water and glycerol to form a mixture; and
(c) incubating the mixture of step (b) under conditions allowing the esterase to catalyze esterification of FFA until FFA levels are reduced below 10 wt %, below 5 wt %, such as below 2 wt %.

In a further aspect the invention relates to a use of the oil product of step c) as a fuel for ships or as feedstock oil for hydrotreated vegetable oil (HVO) production.

### SEQUENCE OVERVIEW

SEQ ID NO: 1 is an esterase obtained from Candida antarctica.

SEQ ID NO: 2 is an esterase obtained from Thermomyces lanuginosus.

SEQ ID NO: 3 is an esterase obtained from Thermomyces lanuginosus.

SEQ ID NO: 4 is an esterase obtained from Chromobacterium viscosum

### BRIEF DESCRIPTION OF THE DRAWINGS

The drawings set forth herein are illustrative of embodiments as provided herein and are not meant to limit the scope of the invention as encompassed by the claims.

FIG. 1 schematically illustrates an exemplary process for production of a crude biodiesel mixture said process comprising as a first step an enzymatic esterification pretreatment of free fatty acids according to the present invention, showing esterification reactor (1), neutralization (2), chemical transesterification (3), feedstock oil (4), enzyme (5), light phase L1 (6), heavy phase H1 (7), light phase L2 (14), heavy phase H2 (8), base catalyst (15), methanol (16), biodiesel reaction mixture (17), crude biodiesel product (18), heavy phase H3 (13), and the optional steps of: drying (9), neutralization of incoming soap (10), methanol addition (11), and recycling of oil from crude glycerin treatment (12).

### DEFINITIONS

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof. In describing and claiming the present invention, the following terminology will be used.

The singular forms “a,” “an,” and “the” include plural referents unless the context clearly dictates otherwise. Thus, for example, reference to “a step” includes reference to one or more of such steps.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context. Similarly, “substantially free of” or the like refers to the lack of an identified element or agent in a composition. Particularly, elements that are identified as being “substantially free of” are either completely absent from the composition or are included only in amounts which are small enough so as to have no deleterious effect on the composition.

Reference to “about” a value or parameter herein includes embodiments that are directed to that value or parameter per se. For example, description referring to “about X” includes the embodiment “X”. When used in combination with measured values, “about” includes a range that encompasses at least the uncertainty associated with the method of measuring the particular value and can include a range of plus or minus two standard deviations around the stated value.

Likewise, reference to a gene or polypeptide that is “derived from” another gene or polypeptide X, includes the gene or polypeptide X.

It is understood that the embodiments described herein include “consisting” and/or “consisting essentially of” embodiments. As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 percent to about 20 percent should be interpreted to include not only the explicitly recited concentration limits of 1 percent to about 20 percent, but also to include individual concentrations such as 2 percent, 3 percent, 4 percent, and sub-ranges such as 5 percent to 15 percent, 10 percent to 20 percent, etc.

Lipid: The term “lipid” refers to phospholipids and their derivatives, triglycerides and derivatives, sterols, stanols, cholesterol, sphingolipids, ceramides, fatty acids, fatty alcohols, glycolipids, proteolipids, lipopolysaccharides, ether-lipids, polar and non-polar lipids and derivatives thereof.

Esterification: The term “esterification” as used herein, refers to a reaction for combining an organic acid such as a fatty acid with any alcohol or polyol such as a glycerol.

Hydrolysis: The term “hydrolysis” as used herein, refers to the reaction of water with an ester to produce an acid and an alcohol.

Alcoholysis: The term “alcoholysis” as used herein, refers to the reaction of an ester with a monohydric alcohol, such as ethanol, butanol, or polyhydric alcohol as glycerol, to produce an ester with a different alkyl group.

Transesterification: The term “transesterification” as used herein, refers to any of the following reactions: alcoholysis, acidolysis and interesterification.

The terms “alkyl” or “alkyl group” is to be construed according to its broadest meaning, to describe a univalent aliphatic compound comprising hydrocarbons.

The terms “glycerol derivatives” and “glycerides” are interchangeably used herein to describe esters, ethers and other derivatives of glycerol in which at least one of the hydrogens, of any of the hydroxyl group attached to the C1, C2 or C3 carbons, is substituted. Examples of glycerol derivatives are: tristearoylglycerol (or tri-Ostearoyl glycerol or glycerol tristearate, or glyceryl tristearate);I,3-benzylideneglycerol (or 1,3-O-benzylideneglycerol); and glycerol 2-phosphate (or 2-phosphoglycerol) among others. If the substitution is on a carbon atom, rather than on the oxygen of the hydroxyl group than the compound may be considered as a derivative of glycerol (e.g., 1,2,3-nonadecanetriol for C16H33CHOH—CHOH—CH2OH, which may be also considered as 1-C-hexadecyl glycerol).

Esterase: The terms “Esterase”, “lipase”, “lipase enzyme”, “lipolytic enzyme”, “lipid esterase”, “lipolytic polypeptide”, and “lipolytic protein” refers to an enzyme in class EC3.1.1 as defined by Enzyme Nomenclature. It may have lipase activity (triacylglycerol lipase, EC3.1.1.3), cutinase activity (EC3.1.1.74), sterol esterase activity (EC3.1.1.13) and/or wax-ester hydrolase activity (EC3.1.1.50). For purposes of the present invention lipase activity (i.e. the hydrolytic activity of the lipase) may be determined with a pNP assay using substrates with various chain length as described in the “Materials & Methods”-section.

Parent or parent Esterase: The term “parent” or “parent Esterase” means an esterase to which an alteration is made to produce the enzyme variants. The parent esterase may be a naturally occurring (wild-type) polypeptide but may also be a variant and/or fragment thereof.

Sequence identity: The relatedness between two amino acid sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

Substrates: Suitable substrates in accordance with the present invention are a broad variety of vegetable oils and fats; rapeseed and soybean oils are commonly used. The substrate may be oil selected from the group consisting of: microbial oil, algae oil, canola oil, coconut oil, castor oil, coconut oil (copra oil), corn oil, cottonseed oil, flax oil, fish oil, grape seed oil, hemp oil, jatropha oil, jojoba oil, mustard oil, canola oil, palm oil, distillers' corn oil, palm stearin, palm olein, palm kernel oil, peanut oil, rapeseed oil, rice bran oil, safflower oil, soybean oil, sunflower oil, tall oil, and oil from halophytes, pennycress oil, camelina oil, jojoba oil, coriander seed oil, meadowfoam oil, seashore mallow oil, or any combination thereof.

Other crops such as mustard, sunflower, canola, coconut, hemp, palm oil and even algae may be used. And derivates of oils and fats, such as palm stearin, used cooking oil, fatty acid distillates, can also be used. The substrate can be of crude quality, used/waste quality, or further processed (refined and/or bleached and/or deodorized). Also, animal fats including tallow, lard, poultry, marine oil as well as waste vegetable and animal fats and oil, commonly known as yellow and brown grease can be used. The suitable fats and oils are mixtures of triglyceride and free fatty acids, commonly seen in waste vegetable oil and animal fats. Partially converted biodiesel, or feedstocks holding some amount of alkyl esters, e.g., FAME, may also be used as feedstocks or part of a blend of feedstocks, such as the olein phase from neutralized biodiesel soap stock. The type of fatty acids in the substrate comprises those naturally occurring as glycerides in vegetable and animal fats and oils. These include oleic acid, linoleic acid, linolenic acid, palmitic acid, steric acid, and lauric acid to name a few. Minor constituents in crude vegetable oils are typically phospholipids, free fatty acids and partial glycerides i.e., mono- and diglycerides. When used herein the phrase “fatty acid residues” refers to fatty acids, either free or esterified as in triglycerides, diglycerides, monoglycerides or fatty acid alkyl esters.

Methanol is mentioned as alcohol in many parts of the text; however, the process of the invention enables use of other alcohols as well, especially ethanol. Therefore, generally, and as the expert in the art will realize based on the examples and prior arts, when methanol is mentioned, the same applies to use of other alcohols and particularly ethanol. Methanol is the standard in industry today and is thus the most relevant alcohol and enables comparison to existing processes, but it is generally derived from fossil sources, with renewable ethanol steadily becoming more widely available and cheaper and thus a relevant alcohol in the longer term. Similarly, when describing FAME (ester with methanol), the same generally applies to alkyl esters, and FAEE (ester with ethanol) in particular.

### DETAILED DESCRIPTION OF THE INVENTION

The present invention relates to an enzymatic pre-treatment process of feedstocks for subsequent production of biodiesel (FAME) by use of base-catalysed transesterification. The pre-treatment comprising an enzymatic esterification of FFA in basically any feedstock oil that can be converted to esterified oil. The feedstock oil having a free fatty acid (FFA) level in the range of 1 to 100 wt %, and preferably 2 to 30 wt % and with esterification reaction taking place in a relatively short amount of time applying economically viable enzyme dosages. The pre-treatment prepares the oil for base-catalyzed transesterification to produce biodiesel. The invention substitutes the more expensive and environmentally problematic sulfuric acid catalyzed esterification and thermal glycerolysis process currently used in the industry.

The present invention, therefore, in a first aspect relates to a process for reduction of free fatty acid concentrations in a fatty acid feedstock by enzymatic esterification with alcohol thereby reducing free fatty acid concentrations to levels compatible with a base-catalyzed biodiesel production process, or as a crude oil product suitable as fuel for ships, or as a feedstock oil for hydrotreated vegetable oil (HVO) production, comprising steps of:
(a) providing fatty acid feedstock comprising more than 1 wt % Free Fatty Acids (FFA);
(b) contacting said fatty acid feedstock with a composition comprising an esterase and at least one component selected from the group consisting of alcohol, alcohol and glycerol, alcohol and water, alcohol and water and glycerol to form a mixture; and
(c) incubating the mixture of step (b) under conditions allowing the esterase to catalyze esterification of FFA until FFA levels are reduced either:
   i) below 10 wt % if a neutralization step follows the enzymatic esterification reaction;
   ii) below 2 wt % if a base-catalyzed transesterification follows the enzymatic esterification reaction without further FFA reduction; or
   iii) below 10 wt %, below 5 wt %, such as below 2 wt % if neither neutralization nor base catalyzed transesterification follows the esterification step c).

The present invention further relates to a use of one or more esterases with no or reduced activity on TAG glycerides (TAG-inactive esterase), which enables selective esterification of FFA. The enzymatic process of the invention may be used as an alternative to common pre-treatment processes, especially acid-catalyzed esterification, but also neutralization and deodorization of feedstocks.

Furthermore, the present invention results in better utilization of excess alcohol, such as methanol stemming from base-catalyzed transesterification.

In one aspect of the present invention, step (a) of the pre-treatment process of the invention provides fatty acid feedstock comprising more than 1 wt % Free Fatty Acids (FFA). Fatty acid feedstock is an oil or fat or derivative thereof, which can be converted into biodiesel. Any such oil or fat being convertible in industry today, using any pretreatment process, will be applicable. The only exception is the FFA level, which must exceed the threshold of 1%, in order to achieve the benefits of the invention.

In one aspect of the present invention, step (b) of said process is contacting said fatty acid feedstock with a composition comprising an esterase and at least one component selected from the group consisting of alcohol, alcohol and glycerol, alcohol and water, alcohol and water and glycerol.

In one embodiment of the present invention, the alcohol is a C1-C5 alcohol, particularly an alcohol is selected from a group consisting of methanol, ethanol, propanol, butanol or mixtures thereof. Preferably the alcohol is methanol or ethanol, most preferably methanol.

In one aspect of the invention, fatty acid feedstock is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, including tallow from pigs, beef and sheep, lard, chicken fat, fish oil, palm oil free fatty acid distillate, soy oil free fatty acid distillate, any other fatty acid distillates, soap stock fatty acid material, yellow grease, used cooking oil, palm oil mill effluent and brown grease or any combination thereof

More specifically, oils and fats generally comprise significant concentrations of MAG, DAG and TAG. Most oils and fats comprise primarily TAG. Other oils and fats, which are somehow degraded or isolated degradation products of oils and fats, can comprise increasing FFA levels at the expense of decreasing TAG levels. TAG becomes DAG, DAG becomes MAG, and so on, through hydrolysis, with FFA as byproduct. This is a natural result of degradation, such as rotting of a slowly harvested ripe palm fruit. So, with increasing levels of degradation, increasing amounts of FFA are formed, and some distribution of MAG, DAG and TAG in the primary remainder, will exist.

Other examples are distillates and other byproducts of traditional refining, which can comprise distributions of FFA, glycerides and unsaponifiable, which are typically very different from extracted oils and fats and degraded qualities thereof.

Another example is soap stock and therefrom acid oil, which can comprise high amounts of phospholipids and derivatives thereof. Partially converted biodiesel, or feedstocks holding some amount of alkyl esters, e.g. FAME, may also be used as feedstocks or part of a blend of feedstocks, such as the olein phase from neutralized biodiesel based soap stock. Such feedstocks are still relevant for the process, which will readily convert FFA to alkyl ester, e.g., FAME, but might require further isolation steps, before or after the enzymatic esterification reaction of this invention, to arrive at a quality that is applicable in further base-catalyzed transesterification. Such relevant further isolation steps may especially be any type of degumming prior to- or following step a, with enzymatic PLA-degumming prior to step a or PLC-degumming following step c being preferable.

The expert in the art will be able to identify further relevant processing steps, which are largely feedstock dependent. Examples are bleaching and distillation, as it will largely depend on the feedstock of choice.

Traditional unit operations such as a drying step prior to the base-catalyzed transesterification may also be part of the process, when warranted by the process design and chosen use of the invention, e.g. when water is purposefully dosed in step b), or otherwise present at significant concentrations during the process of the invention. Water can for example be present in significant amounts in some relevant feedstocks, especially in soap stock or select crude oils.

In one aspect, FFA concentrations in the fatty acid feedstock of step (a) are in the range of 2-30 wt %. Esterification of FFA will lead to water formation, and there is an equilibrium between FFA and alcohol, and water and alkyl ester, e.g., FAME, which limits the reachable FFA concentration at equilibrium. However, any concentration of FFA can be converted in the process, by optimally combining reaction time, esterase dosage, optional use of various means of drying that will be further described, and dosage of methanol and glycerol. As such, pre-drying of the feedstock can be beneficial, because low amounts of water in the process will lead to lower levels of FFA, a well-established fact in the industry.

The components of the mixture of step (c), provided in steps (a) and (b), may be provided in any order and some or all may be pre-mixed in any order. They might also be added at any dosage rate, for example continuously and/or stepwise, that is meaningful within the limits of the desired and feasible reaction time. For example, the components of the composition may be added to the tank in which the fatty acid feedstock is provided, and all components are mixed therein before transportation to the reactor. They may, for example, also be dosed individually or in mixes, and added directly to the reactor at various dosage rates. For example, in a batch reaction, it is beneficial to slowly dose methanol, allowing the esterase to keep alcohol concentrations low through conversion, while it is beneficial to have water and/or glycerol present in significant quantities from time zero. In continuous setups, methanol dosage and concentration in the reactor will reach a steady state level, and the expert in the art will be able to identify optimal dosage rate and quantities.

In one aspect of the present invention, said esterase is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Mucor miehei, Chromobacterium; Candida rugosa lipase; Corynebacterium acnes lipase; Humicola lanuginose, Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas sp, ATCC 21808, Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof.

In one aspect of the present invention, esterase in step (b) is not immobilized.

In one aspect of the present invention, the esterase is added as a liquid, granule, and/or powder.

In one aspect of the present invention, the esterase is a TAG-inactive esterase, or substantially free of TAG-activity. The esterase may also beneficially be substantially free of MAG- and DAG-, while still being active in conversion of FFA to alkyl ester, e.g., FAME. However, for most relevant feedstocks, low activity in conversion of TAG is most important.

In one aspect of the present invention, said esterase is an esterase of SEQ ID NO: 1, SEQ ID NO: 2, SEQ ID NO: 3, SEQ ID NO: 4 or and esterase having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 1, SEQ ID NO: 2, SEQ ID NO: 3 or SEQ ID NO: 4.

In one aspect of the present invention, said esterase has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 1.

In one aspect of the present invention, said esterase has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 2.

In one aspect of the present invention, said esterase has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 3.

In one aspect of the present invention, said esterase has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 4.

The esterase can be any esterase, or combinations of several, with the ability to esterify FFA in the presence of methanol. Such esterase is preferably measurably active for at least 1 hour in a system comprising 11 g methanol in 100 g of refined palm oil in presence of 2 g of water in a pH range of 5-6, adjusted by addition of NaOH and measured in the water phase, and at a temperature of at least 35° C. This is a sufficient test, as many esterases will fail due to a typical loss of thermostability in presence of significant amounts of methanol, and therefore be unsuited for biodiesel pretreatment by the means of this invention. Any esterase that overcomes this difficult environment may be utilized as esterase within the scope of the invention. It is possible to change conditions according to the stability of the esterase, by increasing addition of water and/or glycerol, or reducing the dosage of methanol, however, such changes may negatively affect the obtainable FFA level at equilibrium.

Most enzymes with esterase activity will typically catalyze hydrolysis of MAG, DAG and TAG to FFA in presence of water or transesterification to alkyl esters in presence of alcohol.

Esterase of SEQ ID NO: 2 and 3 are examples of esterases having near-equal preference in reacting MAG, DAG and TAG. MAG, DAG and TAG, however, have increasingly larger sizes, and as the activity of enzymes typically comes from a very specific part of the enzyme called the active site, which may not be easily accessible, some enzymes will show a size-dependent specificity between MAG, DAG and TAG. Therefore, larger or more sterically hindered molecules may not easily fit in the active site, and reaction will be slower or non-existent for some otherwise theoretically convertible reactants. In case of SEQ ID NO: 1, the size of TAG is a significant sterical hindrance for entrance into the active site and subsequent reaction, meaning the enzyme will only convert TAG at a very low rate. At increasing dosages of SEQ ID NO: 1, the low activity on TAG can be mitigated and result in measurable activity on TAG. Therefore, definition of MAG, DAG and TAG-activities of lipases with sterically hindered active sites will not be binary, but rather relative between the glyceride species.

The term “TAG-inactive” as mentioned in the present invention is specifically meant to encompass enzymes with significant difference in activity between esterification of FFA, the relevant reactant, relative to transesterification of TAG, the largest theoretically convertible compound in the system. Lack or significantly reduced activity on TAG may also be determined by the relative activity in transesterification of MAG relative to TAG, or DAG relative to TAG. Specifically, if the initial rate of conversion of FFA is at least double the rate of conversion of TAG, the enzyme exhibits preferential activity on FFA, and is preferable for use in the invention relative to enzymes with substantially similar initial rate of reaction on FFA, MAG, DAG and TAG.

Enzymes may also exhibit reduced activity on more than one glyceride species, such as low- or no activity on both DAG and TAG, or on MAG, DAG, and TAG altogether. High activity in esterification of FFA relative to the most-abundant glyceride species, typically TAG, is most important, and is covered by the term “TAG-inactive”. For a TAG-inactive esterase, preferably less than 10%, preferably less than 5%, more preferably less than 2% and most preferably less than 0.5% of the triglycerides present in the oil is converted during reaction.

Further, the esterase is preferentially active in esterification of FFA rather than transesterification of MAG, DAG and TAG. A TAG-active esterase will require more methanol and reaction time because methanol and esterase activity are consumed and occupied, respectively, in conversion of TAG rather than FFA. MAG and DAG conversion is of lesser importance because of their relatively low concentrations in most fatty acid feedstocks.

Most preferably, at least the esterase of SEQ ID NO: 1, or variants thereof, is used in the process, as it exhibits the above preferable properties, being preferentially active in conversion of FFA rather than TAG, while being extraordinarily tolerant regarding presence of methanol. It may also be that at least the esterase of SEQ ID NO: 2 or 3 may be used, which are sufficiently methanol tolerant, but are largely equally active in reaction of TAG and FFA. Best choice of esterase will primarily be a matter of esterase cost, acceptable reaction time, and methanol tolerance of the esterase limiting the obtainable FFA concentration through applicable conditions in terms of dryness and methanol concentration during reaction.

The esterase may be applied in any formulation or form. Typically, free or immobilized esterases are applied in the industry today, but other relevant formulations are e.g. powders and slurries. The invention is most preferably practiced using free esterase applied in liquid or other easily handled formulation, because such formulations are usually safest and cheapest for the producer and need not be separated from the product by filtration after reaction.

In an embodiment of the present invention, the total amount of said esterases added is within the range 0.1-50000 mg enzyme protein (EP)/kg of oil. Preferably 0.1-200 mg enzyme protein (EP)/kg of oil in cases where a liquid enzyme formulation is used, and preferably 500-50000 mg enzyme protein (EP)/kg of oil in cases where an immobilized enzyme formulation is used.

In one aspect of the present invention, the total amount of esterase is within the range of 5-8000 ppm (wt enzyme protein/wt of substrate).

In one aspect of the present invention, the total amount of said non-immobilized esterase is within the range of 5-1000 ppm (wt enzyme protein/wt of substrate).

Alcohol, preferably methanol addition will be in the range of 0-3 molar equivalents, and preferably 0-2 molar equivalents based on the total number of fatty acids in the mixture, if methanol dosage is controlled directly. In case methanol is only dosed through the preferred use of the heavy phase stemming from base-catalyzed transesterification, it may not be necessary to dose additional methanol, because a significant amount will be present in the heavy phase mixture. The optional choice of additional methanol dosage will depend on the amount of FFA in the feedstock.

Water addition will be in the range of 0-20% (wt/wt of oil feedstock), and preferably as low as possible depending on the stability of the chosen esterase. Most preferably, no water is directly added.

Glycerol addition will be in the range of 0-20% (wt/wt of oil feedstock). Preferably, glycerol stemming from the base-catalyzed transesterification is added. This amount will typically be in the range of 0.5-14% (wt/wt of oil feedstock) depending on the feedstock type and original FFA level. A 100% TAG feedstock of mean FFA molar mass of 282 g/mol (oleic acid, C18:1), will yield around 11% glycerol, while a feedstock containing primarily FFA will yield little glycerol. Procured glycerol or glycerol from unrelated processes in the plant may also feasibly be used.

A general and critical fact is that methanol destabilizes most esterases, while water and glycerol can beneficially be used to dilute methanol to manageable levels, allowing for efficient reaction. There are many variations to dosage of the components, and while most are applicable, the best choice will be identifiable by the expert in the art based on the design of the plant.

Additionally, some or all the individual components of the composition may be provided by the heavy phase originally stemming from the base-catalyzed transesterification reaction. Said heavy phase is preferably partially or fully neutralized or even acidified by use of an acid, such as organic and/or inorganic acids, e.g. hydrochloric acid, sulfuric acid, citric acid or phosphoric acid. This is done to reduce pH to a range where the esterase is optimally effective. This range is typically in the range of 3.5 to 8, and preferably within a range of 4-6, but will vary depending on the esterase(s) of choice.

In one aspect of the present invention, step (c) of the process is incubating the mixture of step (b) under conditions allowing the esterase to catalyze esterification of FFA until FFA levels are reduced to levels compatible with downstream process steps, which eventually will result in FFA levels in the light phase going into the base catalyzed transesterification reaction of below 2 wt %, but preferably below 1 wt %, 0.5 wt %, 0.25 wt %, such as 0.1 wt %.

A preferred embodiment of the invention builds on the realization that FFA does not have to be lower than 1 wt % to enable beneficial use of the invention. Particularly, when a neutralization step, which preferably is alcoholic neutralization is applied in case FFA levels after esterification are reduced to below 10 wt % of FAA. In this case a separation step may be applied before the neutralization step and/or after. The FFA content in step (c) is reduced to less than 10%, but preferably to less than 6 wt %, 4 wt %, 2 wt %, 1.5 wt %, such as less than 1.25 wt % of FFA. Particularly, the FFA content in step (c) (i) is reduced to the range of 0.5-8 wt %, 0.75-4 wt %, such as 1-2 wt %. Lower FFA levels are preferable, because separation of oil and soap becomes increasingly difficult with higher amounts of FFA and thereby soap present. However, there is no significant yield loss associated with soap stock formation when a preferable neutralization and recycling of the fatty material is conducted.

In another aspect of the invention, it is also possible to skip the neutralization step, and instead follow step (c) with based catalyzed transesterification. In this case less than 2 wt % FFA is acceptable if no neutralization is done, but at the expense of additional base-catalyst dosage in the transesterification step. Therefore, in one embodiment of the present invention, the FFA content in step (c) ii) is reduced to less than 1.5 wt %, 1.0 wt %, 0.75 wt %, 0.5 wt %, such as less than 0.25 wt %.

It is also conceivable that one would choose to conduct a neutralization step using a cheap base because this would reduce loss of base-catalyst in transesterification through use of a cheaper base. Such dosage of cheap base, such as NaOH, would preferably not require a further separation step between addition of the base and further base catalyzed transesterification. For example, one could use step (c) (ii), obtain 2 wt % FFA, add the mixture to the base-catalyzed transesterification reactor, then add NaOH to neutralize the soap, and only then dose the expensive methylate catalyst, resulting in reduced loss of the valuable catalyst. One may also choose to dose the NaOH prior to adding the neutralized, but un-separated, mixture to the base-catalyzed transesterification reaction, which would especially be preferable in a continuous reaction setup.

In one embodiment of the present invention, the product of step (c) (i) is optionally separated into a first light phase L1 (6) comprising esterified oil and FFA below 10 wt %, and a first heavy phase H1 (7) comprising glycerol before the neutralization step. Alternatively, separation can be performed after the neutralization step, or both separation steps can be applied. The effect of the neutralization step is reduction of FFA to levels compatible with base-catalyzed transesterification by neutralization. Especially use of an alcoholic neutralization step is preferred. Including a neutralization step allows for higher levels of FFA in the treated feedstock of step (c). This reduces required reaction time significantly and brings robustness to the process. Alcoholic neutralization utilizes the alkalinity of the heavy phase stemming from base-catalyzed transesterification. It may further be beneficial to include additional dosage of e.g. NaOH or KOH in the alcoholic neutralization step, in case there is insufficient residual alkalinity in the heavy phase to bring down FFA levels sufficiently from the levels achieved in step (c).

In one embodiment, the first light phase L1 (6) or the reaction mixture of step (c) (i) is neutralized (2), preferably by alcoholic neutralization, and with optional further addition of alcohol and/or base, resulting in an FFA level below 2 wt %, particularly below 1 wt %, particularly below 0.5 wt %, more particularly below 0.25 wt %, such as below 0.1 wt %.

In one embodiment, the neutralization (2) is followed by separation into a second light phase L2 (14) comprising neutralized oil, and a second heavy phase H2 (8) comprising neutralized glycerol.

In one embodiment, the neutralized oil in the second light phase L2 (14) is reacted in a base catalyzed transesterification reaction (3) in presence of alcohol resulting in a biodiesel reaction mixture comprising alkyl ester, e.g., FAME.

In another embodiment, the esterified oil in the first light phase L1 (6) is reacted in a base catalyzed transesterification reaction (3) in presence of alcohol resulting in a biodiesel reaction mixture comprising alkyl ester, e.g., FAME. This is especially the case for step (c) (ii), having resulted in FFA levels below 2 wt % but preferably below 1 wt %, 0.5 wt %, 0.25 wt %, such as 0.1 wt %, requiring no further FFA reduction, and thus there is no need for a neutralization step.

In one embodiment of the present invention, the biodiesel reaction mixture comprising alkyl ester, e.g., FAME is separated into a third light phase L3 (18) comprising a crude biodiesel/alkyl ester, e.g., FAME product, and a third heavy phase H3 (13) comprising alkaline crude glycerol.

In a preferred embodiment of the invention, the heavy phase stemming from the base-catalyzed transesterification is at least partially neutralized or even acidified by use of neutralization.

Said heavy phase originally stemming from base-catalyzed transesterification may additionally have been flashed or dried, with or without having been neutralized in a neutralization step. Such steps are already existing in most biodiesel production plants today. This means lower amounts of both methanol and water in said heavy phase, with the reduced amount of water being beneficial.

In one embodiment of the present invention, the third heavy phase H3 (13) is recycled back to the neutralization step (2) of the process. The heavy phase of the neutralization step (2) is then optionally added in step (b).

In one embodiment of the present invention, the reaction mixture of step (c) (ii) is reacted in a base catalyzed transesterification reaction (3) in presence of alcohol resulting in a biodiesel reaction mixture comprising alkyl ester, e.g., FAME.

After separation of the light phase (L1) and the heavy phase (H1) this allows for reuse of enzyme, drying and recirculation of some or all of the heavy phase, and can thus be employed to bring down cost, reaction time and/or FFA levels through dryness achieved in recycling of dry glycerin.

Preferably, the contents of the mixture step (c) (ii) are dry, and a further drying step between step (c) and the base catalyzed transesterification reaction may be applied.

In one embodiment of the present invention, the reaction mixture of step (c) (ii) is first separated to yield a light phase L1 (6), which is then reacted in a base catalyzed transesterification reaction (3) in presence of alcohol resulting in a biodiesel reaction mixture comprising alkyl ester, e.g., FAME. Preferably, the light phase of step (c) (ii) is dry, and a further drying step between step (c) and the base catalyzed transesterification reaction may be applied.

In one embodiment of the present invention, the second heavy phase H2 (8) and/or third heavy phase H3 (13) is recycled back to step (b) of the process.

In one embodiment of the present invention, the second heavy phase H2 (8) or third heavy phase H3 (13) is recycled back to the reaction mixture of step (b) and the amount of heavy phase comprises at least 2% (wt/wt) of the fatty acid feedstock.

In one embodiment of the present invention, the process comprises acidulation of first heavy phase and further separation of the acidulated first heavy phase into oil and crude glycerin and recycling to the reaction mixture in step (b).

In one embodiment of the present invention, e.g., when alcoholic a neutralization step is not included, comprises adding acid to neutralize the second heavy phase H2 (8) or third heavy phase H3 (13) before or during recycling to step (b) of the process.

In one embodiment of the present invention, the said process comprises drying of the mixture in step (b) prior to reaction in step (c).

It is generally advisable, but often not required, to adjust pH in the mixture by addition of an acid or base or combination of both, which forms a stabilizing buffer. Target pH is within 3.5 and 8, and preferably within 4-6, but depends on the esterase of choice. Typically, addition of sodium hydroxide in amount between 5 and 1000 ppm, and especially between 5 and 100 ppm is observable in industry. Sodium hydroxide addition is most often practiced for conversion of acidic oils and fats, especially lauric oils, where an increase in efficiency of some esterases is obtainable as a result, even though the small dosage of sodium hydroxide will not significantly impact the composition of the mixture.

Dryness of the reaction mixture is to some extent preferable, especially when desired FFA levels are below 1 wt % or even below 0.5 wt %. In such cases pre-drying of glycerol, and use of dry methanol and fatty acid feedstock is advisable. As is further described below, dryness can be achieved during reaction by several means, yielding the same result.

In one aspect of the present invention, drying is used especially for reduction of the methanol concentration in the recycled heavy phases H2 or H3. Excess methanol (as shown in example 7) in the heavy phase stemming from base-catalyzed transesterification can lead to no reaction. Water addition or glycerol accumulation by recycling of glycerol around step c, can mitigate the negative effect, but drying off of some amount of the incoming methanol will be preferable for economical and environmental reasons.

In one aspect of the present invention, drying comprises separating the heavy phase from the reaction mixture, drying the heavy phase, and adding some or all back into the reaction mixture.

Ideal conditions according to the invention are those of high methanol, low water, and preferably glycerol, because it results in the lowest FFA concentration through chemical equilibrium. Glycerol acts to dilute methanol thereby protecting the esterase, further, glycerol reduces the chemical activity of water thereby bringing down the achievable FFA level at equilibrium, and finally, in case low water concentrations are present, glycerol acts to form a heavy phase from which the esterase can react. Often, esterases require a heavy phase in order to be active, especially when applied in free form, and lipases are for example often interracially activated, thus exhibiting no meaningful activity without presence of either a water or glycerol heavy phase. Such conditions will be difficult for most esterases, however, a benefit of the invention is short reaction time, which may enable the possibility of using esterases that are otherwise too intolerant regarding presence of methanol to run in the state-of-the-art enzymatic process described above, because they need not be active for an extended period of time.

The Esterification step (c) of the invention is preferably performed at temperatures in the range of 20-90° C., such as 25-85° C., particularly 30-80° C.

Reaction temperatures will be in the range of 20-90° C., preferably in the range of 25-85° C., and most preferably in the range of 30-80° C. The choice of temperature will depend especially on the thermostability and methanol tolerance of the choice of esterase.

In case FFA must be reduced to <1 wt % during esterification in step (c), it will be necessary to operate in a difficult environment of high methanol and low water, which will affect most esterase's stabilities severely. This can be mitigated by operating at lower temperatures, but at the cost of rate of reaction. A faster reaction is achievable by use of higher temperatures and higher amounts of water present, which conversely will negatively affect achievable FFA concentrations.

The total reaction time in the pretreatment step of the invention will be below 48 hours, below 24 hours, preferably below 16 hours and most preferably below 12 hours. However, reaction times below 8, 6, 4 hours and even about 2 hours is possible to achieve according to the invention. Reaction time will depend especially on the dosage and choice of esterase, with TAG-inactive esterases bringing faster reaction by not being occupied in meaningless conversion of TAG. Further, addition of water typically increases reaction rate indirectly by allowing optionally faster dosage of optionally more methanol, but at the expense of higher achievable FFA concentrations at chemical equilibrium. Similarly, dosage and dosage rate of methanol, which depends on the esterase tolerance for methanol at the chosen reaction conditions, may be the limiting factor regarding reaction rate. Finally, the required FFA concentration after reaction can beneficially be set higher than the chemically obtainable concentration, which will significantly shorten reaction time, especially when a neutralization step follows the reaction. For example, chosen conditions could allow for 0.5 wt % FFA at chemical equilibrium, but reaction may beneficially be stopped at 1.5 wt % FFA, followed by optional but preferable neutralization, thus lowering the required reaction time.

In one aspect of the present invention, reaction may be conducted in any combination of batch and/or semi batch and/or continuously. Reactor sizes may vary according to optimal design and cost of the plant.

Rate of reaction is typically high initially but slows down when equilibrium FFA levels are approached. Therefore, the optimal plant design, especially regarding reactor sizes, footprint of the process, and the like may be optimized by a carefully choosing reactors of different modes of operation and of different sizes. This choice may vary depending on practicalities especially in existing plants, where footprint is usually an issue due to space constrictions.

As such, the optimal plant design may involve any combination of reactor sizes and modes of operation. A preferred design involves one or two CSTR reactors in series.

Further, addition of especially esterase and methanol may be split equally or non-equally between different reactors depending on the setup. For example, it may be beneficial to dose additional esterase at later stages of the reaction, if conditions are difficult, leading to destruction of enzyme activity as reaction progresses. Similarly, dosing of methanol may be beneficially split between reactors to reduce the negative effects of methanol upon the enzyme in early stages of the reaction.

In one embodiment of the present invention, separation steps are gravitationally based, conducted by means such as centrifugation or setting.

In one embodiment of the present invention, enzyme is isolated and reused as an emulsion phase. Often, an emulsion phase exists between the light oil phase and heavy water/glycerol phase, and this emulsion phase will hold a concentrated part of the enzyme activity, which may feasibly be reused. Such isolation of enzyme-rich emulsion phase may e.g. happen by simple settling, use of tri-phase separators, or through reuse of the material, which is obtained when centrifuges release their accumulated solids and middle phase emulsion.

In another embodiment of the present invention, membranes are used as a means of separation. Membranes may allow for separation of small molecules like water, methanol and glycerol from larger molecules such as enzyme protein and fatty acid-based molecules. Membranes may also be used as a means of drying, when water may preferentially permeate the membrane of choice.

An optional embodiment of the invention regards the use of drying during reaction and/or between two or more reaction steps. As described above, lowering the concentration of water will lower the obtainable FFA concentration determined by the chemical equilibrium between the reactions of FFA and methanol versus alkyl ester, e.g., FAME and water. Lowering the concentration of water is therefore beneficial, and in some cases required, if the invention is applied especially with a low target FFA concentration of e.g. 0.75 wt % after treatment. Drying during reaction can beneficially be combined with drying of input streams of steps (a) and (b) as mentioned above.

Drying may be done by any conceivable means. Preferably, the esterase of choice will tolerate the conditions in which drying is conducted, but it is not a requirement because more esterase can be added to make up for any loss. The most preferred means of drying is a recirculation loop over a connected, preferably flash, dryer with an optional change of temperature accompanying the drying step and required drying efficiency. Between one or more reactors in series, there may also be drying steps where the whole mixture or the heavy phase is dried before entering the next reactor. It may also be achieved through less conventional means such as addition of dry powders, salts, beads and the like, which will absorb moisture, and which can be removed from the liquid phases after reaction by means of e.g. filtration. Further, membranes may be used to remove the moisture.

Dryness may also be achieved by drying of just one of the liquid phases of the reaction mixture following a separation step, before blending some or all the dried material back into the reaction stage.

Finally, drying can be applied continuously or in steps. It may be beneficial economically to let the reaction run until equilibrium, before applying the chosen procedure of drying, and letting reaction run again. This may save costs associated with drying.

The options are many, and the above is not exhaustive and only meant as an indication of which means of drying may be applicable. An expert in the art, or even a chemical engineer without knowledge within the field of biodiesel, will be able to identify a good choice of drying process depending on the specific design of the plant. For example, many existing plants will have vacuum systems available with excess capacity, such as the glycerol drying or biodiesel product drying processes in the downstream processing steps following the scope of the invention.

A further benefit of employing alcoholic neutralization is the fact that it will significantly reduce concentrations of phosphorus in the feedstock oil. Many relevant feedstock oils comprise significant concentrations of phosphorus as phospholipids. Such phospholipids are typically removed in one or more pretreatment steps in upstream preparation of such feedstocks prior to base-catalyzed transesterification. However, a neutralization step following reaction step (c) may beneficially reduce the required extent of phosphorus removal upstream.

A separation step preferably follows the optional neutralization step. This especially avoids water entering the base-catalyzed transesterification reaction. However, drying of the mixed neutralized oil and heavy phase may be used to avoid a separation step. If alcoholic neutralization is conducted, then separation of heavy phase from the treated oil phase is especially preferable to allow for usage/recycling of the heavy phase in the step (c) reaction with all the benefits mentioned above.

For example, in case step (c) i) is not utilized, the heavy phase of step (c) may be transferred with the treated feedstock directly into either neutralization or base-catalyzed transesterification. This may remove a separation step, which can be expensive in case of procurement of industrial centrifuges. However, significant amounts of water may be present after the step (c) reaction, and in such cases, it would not be preferable to add the whole step (c) mixture directly into the base-catalyzed transesterification reaction. A drying step in between may solve this problem. The possibility and usefulness of employing this option also depends on the amount of FFA after reaction step (c) and the required FFA level. Generally, if FFA is above 2 wt %, then further FFA reduction is required, as it would be suboptimal in terms of cost to add the mixture of step (c) directly into the base-catalyzed transesterification, leading to significant consumption of catalyst. But if FFA is below 2 wt % it may not be necessary to reduce FFA levels further, and addition of NaOH or KOH just prior to the base-catalyzed transesterification may alleviate the costly consumption of base catalyst related to FFA in the feedstock of the base-catalyzed transesterification step.

In one aspect of the present invention, the enzymatic reaction does not result in total glycerin (also called bound glycerin or glycerol) concentrations below 0.25 wt %, preferably not below 0.5 wt % and most preferably not below 1 wt %. Low total glycerin after reaction, would mean high conversion of glycerides, if glycerides were initially present at high concentrations. Since conversion of glycerides will take up precious enzyme activity and consume methanol, reduction of total glycerin concentrations below the initial level of e.g. 11% (wt/wt oil) in case of a pure triglyceride oil, is a measure of unnecessary reaction happening. Use of an enzyme with low conversion of glycerides, leading to high residual total glycerin concentrations following reaction, will yield a superior result with a lower dosage of both enzyme and methanol, than is possible using an enzyme capable of converting glycerides significantly.

In one embodiment of the present invention, the part or all of the first heavy phase H1 (7) is recycled into the mixture of step (b), or alternatively in case the first separation step is not performed, then part or all of the second heavy phase H2 (8) is recycled into the mixture of step (b).

In one aspect of the present invention, the FFA concentration in the feedstock does not exceed 90 wt %, 80 wt %, 70 wt %, 50 wt % and particularly 30 wt % or even 15 wt %.

In one aspect of the present invention, the FFA content in the feedstock is at least 1.5, at least 2, at least 2.5, at least 3, at least 5, such as at least 10 wt %.

In one aspect of the present invention, the FFA content in the feedstock is in the range from 1-100 wt %, 1-70 wt %, 1-60 wt %, 2-50 wt %, 2-30 wt %, such as 3-10 wt %.

In one aspect of the present invention, transesterification is conducted on the enzymatic treated feedstock. The transesterification is well known in the art of biodiesel production, and the invention does not result in significant changes to that, and the authors refer to prior art publications for complete description of a base-catalyzed transesterification. The FFA concentration in the pretreated feedstock must at least be below 2 wt % following the prior steps. Preferably the FFA concentration is below 1 wt %, such as 0.75 wt %, 0.5 wt % and most preferably below 0.25% or even 0.1 wt %.

In a particularly preferred aspect of the present invention, the process comprises the steps of:
(a) providing a fatty acid feedstock comprising free fatty acids (FFA) in the range from 2 wt % to 30 wt %;
(b) contacting said fatty acid feedstock with a composition comprising a substantially TAG-inactive esterase and alcohol and glycerol; and
(c) incubating the mixture of step (b) under conditions allowing the esterase to catalyze esterification of FFA until FFA levels are reduced below 2 wt %; and further comprising
(d) separating the product of step (c) into a first light phase L1 and a first heavy phase H1; and
(e) neutralizing the residual FFA in the light phase L1 in an alcoholic neutralization step (2); and
(f) separation of the neutralization product of step (e) in a second light phase L2 (14) and a second heavy phase H2 (8); and
(g) base catalyzed transesterification of the light phase L2 (14) to produce a crude biodiesel reaction mixture.

In one embodiment, some or all of the alcohol, particularly methanol, and/or glycerol stems from the alcoholic neutralization step (2).

In another embodiment, the reaction time of step (c) does not exceed 8 hours.

In another embodiment, at least 1%, preferably at least 4% total glycerin remains unconverted after step (c).

In another particularly preferred aspect of the present invention, the process comprises the steps of:
(a) providing fatty acid feedstock comprising more than 1 wt % Free Fatty Acids (FFA);
(b) contacting said fatty acid feedstock with a composition comprising an esterase and alcohol and glycerol; and
(c) incubating the mixture of step b) under conditions allowing the esterase to catalyze esterification of FFA until FFA levels are reduced below 1 wt %; and in case the amount of water in the incoming streams and FFA in the feedstock results in water concentrations that leads to FFA concentrations exceeding 1 wt % at equilibrium, then at least one of the following steps is applied:
   (c) (i) Drying the reaction mixture, and/or
   (c) (ii) Drying incoming streams of oil, and/or glycerol, and/or
   (c) (iii) Separating the light phase from the heavy phase, drying the heavy phase, and recycling at least part of the dried heavy phase back into step (c);
(d) separating the product of step (c) into a first light phase L1 (6) and a first heavy phase H1 (7), which optionally is combined with step (c) (iii) above; and
(e) base catalyzed transesterification of the second light phase L2 to produce a crude biodiesel reaction mixture.
The alcohol is preferably methanol.

In one embodiment, the esterase is a TAG-inactive esterase, or is substantially free of TAG-activity.

In one embodiment, the esterase is selected from an esterase having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 1.

In one embodiment, the reaction time of step (c) does not exceed 48 hours, and preferably does not exceed 24 hours, and most preferably does not exceed 12 hours.

In another embodiment, at least 5% (wt/wt of oil) total glycerin is present in the oil after step (c).

In another embodiment, at least 50% of the original total glycerin remains unconverted after step (c).

In another embodiment, at least the glycerol is provided by neutralization of the third heavy phase (H3).

In another embodiment, the total amount of water present is kept below 1 wt % of the total reaction mixture mass.

In another embodiment, the FFA level is reduced to below 0.75 wt %, particularly below 0.5 wt %, and most particularly below 0.25 wt %.

In another embodiment, the FFA concentration in the feedstock does not exceed 90%, 80%, 70%, 50% and particularly 30% or even 15%.

In another embodiment, the FFA concentration in the feedstock does not exceed FFA content in the feedstock is at least 1.5, at least 2, at least 2.5, at least 3, at least 5, such as at least 10 wt %.

In another embodiment, the FFA content in the feedstock is in the range from 1-100 wt %, 1-70 wt %, 1-60 wt %, 2-50 wt %, 2-30 wt %, 3-20 wt %, such as 3-10 wt %.

The process may bring a reduction in the required total dosage of methanol in the combined biodiesel production process including pretreatments. This may be achieved through improved utilization of methanol by reuse of said methanol by use of the glycerol phase stemming from step (c) of the process.

Further, step (c) may be run at sub-stoichiometric dosages of methanol, based on the total number of fatty acids in the feedstock, with good effect, leading to a reduction in the required dosage of methanol.

In base-catalyzed transesterification, there is typically a high excess of methanol relative to the stoichiometrically required dosage. But with the preesterification treatment of step c., that dosage may be reduced. This is especially the case when step (c) is run in presence of an esterase that can catalyze transesterification of the glycerides, while simultaneously reducing FFA through esterification. This is not a preferred embodiment of the invention, however, as significant glyceride activity results in increased reaction time, and a need for dryness and more methanol in the enzymatic reaction step.

The process as described in this invention, is primarily meant as a means to pretreat feedstocks prior to chemical transesterification. The full process as described according to the invention, will result in a biodiesel product of good quality and with applicability in standard diesel engines as described in the prior art. However, it turns out that the enzymatic pretreatment reaction of claim 1 c) also has utility as a means for FFA reduction outside the narrow field of standard quality biodiesel production, e.g. as described in the European EN14214 standard. For example, within the field of shipping, cheap fuels of generally lower quality can feasibly be used. This is because the engine designs allow for combustion of fuels, which are typically not applicable to automobile diesel engines. However, even though fuels of generally lower quality may be used, the concentration of acid will directly impact the lifetime of such ship engines through the significant degree of corrosion, especially at combustion temperatures. There are several types of low-quality byproduct oils on the market, such as used cooking oil, soapstock/acid oil, palm oil mill effluents, and the like, which would in principle be acceptable fuels in ship engines, but which generally contain too high concentrations of free fatty acids to acceptably employ. For such feedstocks, the invention may be used as a means for reducing FFA concentrations to acceptable levels, which are at time of writing not established by standards, but which are likely to be less than 10 wt % FFA, such as 5 wt % or 2 wt %, and preferably near-zero.

Similarly, as another example, within the field of hydrotreated vegetable oils (HVO), the process of fully reducing renewable fatty acids into aliphatic hydrocarbons similar to fossil fuels, there is a need for low-acidity fatty acid feedstocks from cheap, waste-based sources. The problems are similar to those of ship engines, where corrosion is the main issue. The end-uses of HVO fuels are similar to fossil fuels, meaning a wide range of applications, as they can be transformed by various processes such as cracking and isomerization to yield products suitable for airplane, automobile and lubrication uses as examples.

Therefore, the invention may be used as a means for pretreating fatty acid feedstocks to low concentrations of FFA. The process of the invention may be employed as the only process step before direct use as fuel following the esterification step according to the process of the invention, without significant further downstream processing. The process of the invention may also be employed as only one of a number of processing and/or reaction steps in conversion of materials containing fatty acids, meaning significant pre- and post-treatment steps may be employed before utilizing the finished product as fuel or treated feedstock.

Further, the process of the invention may be employed before downstream processing, such as HVO, that may not involve transesterification of unreacted glycerides, but requires low-acidity feedstocks. For example, soap stock, the byproduct of FFA neutralization in chemical refining of e.g., soybean, rapeseed and sunflower oils, is a suitable fatty acid-based feedstock for shipping and HVO. Conversion of soap stock to shipping fuel involves at least 1) a neutralization step, converting soaps to FFA by acid addition, 2) degumming of phospholipids, which stem from the original virgin oil, e.g., soybean, and 3) enzymatic esterification of FFA as described in the process of the invention. Steps 2 and 3 can be combined by addition of PLA or PLC phospholipase in the process of the invention but is not necessary and may not be economical either. Additionally, depending on the origins of the soap stock, and degree of blending with waste oils of various qualities (a somewhat common practice in the industry is to mix used cooking oil, soap stock, palm oil mill effluents, lecithin and the likes), there might be a need to bleach, deodorize, distill, wash, further neutralize, and so on. As should now be clear to the expert in the art, the process of the invention will not always be able to address the full scope of processing required to arrive at acceptable low acid fuels and feedstocks. But it will be a valuable new tool in the industry, especially because it allows for selective conversion of FFA into non-acidic esters, without significant conversion of glycerides, which, among other benefits, significantly reduces the alcohol consumption for esterification and therefore the cost.

Therefore, in an additional aspect the invention relates to an enzymatic process for producing an oil suitable as fuel for ships, and/or as an oil feedstock for further downstream processing not involving transesterification, comprising the steps of:
(a) providing fatty acid feedstock comprising more than 1 wt % Free Fatty Acids (FFA);
(b) contacting said fatty acid feedstock with a composition comprising an esterase and at least one component selected from the group consisting of alcohol, alcohol and glycerol, alcohol and water, alcohol and water and glycerol to form a mixture; and
(c) incubating the mixture of step (b) under conditions allowing the esterase to catalyze esterification of FFA until FFA levels are reduced below 10 wt %, below 5 wt %, such as below 2 wt %.

In one embodiment the alcohol is methanol or ethanol, particularly ethanol.

The oil product, if not intended as fuel for ships, may be further processed, e.g., the process may further comprise a hydrotreatment step, yielding aliphatic hydrocarbons. The resulting hydrotreated vegetable oil (HVO) may be used as HVO fuel.

In a further embodiment the invention relates to a use of the oil product produced by the process of the invention as a fuel for ships or as a feedstock oil for hydrotreated vegetable oil (HVO) production.

### Further Advantages of the Present Invention

Environmental improvements that generally follow use of enzymes when substituting traditional chemical processes. Examples are reduced temperatures, ambient pressures, employment of a renewable and biodegradable catalyst, applicability of simple steel alloys with low amounts of expensive and CO₂-intensive metals, and so on.
Significantly reduce methanol excesses by preferred reuse of optionally wet (non-rectified) methanol, and by preferable use of largely TAG-inactive esterases, compared to both state-of-the-art uses of enzymes as well as acid-catalyzed esterification pretreatments.
Avoid use of acid-catalyzed esterification as pretreatment prior to base-catalyzed transesterification.
   The scope of the invention allows for use of existing acid-catalyzed esterification equipment with little requirement for retrofitting due to the high rate of reaction. This enables substitution of existing acid-catalyzed esterification plants, and a positive environmental and economic benefit as a result.
   Acid esterification may cause significant color formation, requiring post-bleaching or post-distillation of the biodiesel. Use of the invention will enable avoiding this issue, as color is largely unchanged by the process.
Avoid use of neutralization as pretreatment prior to base-catalyzed transesterification. This will reduce yield losses and/or use of base and acids in soap stock recycling.
Avoid use and especially procurement of deodorization as pretreatment prior to base-catalyzed transesterification. Deodorizers run at >220° C. typically, and the environmental benefit in running a near-ambient temperature process is thus significant.
Significantly reduce reaction time relative to state-of-the-art uses of enzymes within the field of biodiesel processing.
   Today, enzymes are typically used for full conversion of waste-oil feedstocks, typically with high FFA levels, in standalone processes without base-catalyzed transesterification. They are also used to convert the soap stock (acidulated, stemming from neutralization above) or fatty acid distillate (from deodorization above), rather than the main stream, followed by blending-in of the crude enzymatic biodiesel back into the main stream and subsequent base-catalyzed transesterification. In both cases, conversion into FAME is largely complete, with complete glyceride conversion as a result.
   The invention allows for a quick reaction with low glyceride conversion.
   And the invention may be economically applied on the main stream rather than side streams or waste oils. This allows for employment of this environmentally benign process on all feedstocks rather than a select few, or side streams only.
Blending-in of high-FFA feedstock of waste quality into higher-quality feedstocks, will become feasible to a much larger extent by use of the present invention. This is especially the case in plants where neutralization or deodorization would be the alternative today. This may allow for productivity increases in conversion of used cooking oil as an example in some operations today.
Enables FFA reduction by specific conversion. This means shipping fuel and HVO feedstock with low acidity may be produced at low cost relative to the cost of producing fully converted biodiesel, which requires full transesterification as further processing step.

The invention is further described in the following numbered paragraphs.
1. A process for reduction of free fatty acid concentrations in a fatty acid feedstock by enzymatic esterification with alcohol thereby reducing free fatty acid concentrations to levels compatible with a base-catalyzed biodiesel production process, or as a crude oil product suitable as fuel for ships or as a feedstock oil for hydrotreated vegetable oil (HVO) production, comprising steps of:
   (a) providing fatty acid feedstock comprising more than 1 wt % Free Fatty Acids (FFA);
   (b) contacting said fatty acid feedstock with a composition comprising an esterase and at least one component selected from the group consisting of alcohol, alcohol and glycerol, alcohol and water, alcohol and water and glycerol to form a mixture; and
   (c) incubating the mixture of step (b) under conditions allowing the esterase to catalyze esterification of FFA until FFA levels are reduced either:
      i) below 10 wt % if a neutralization step follows the enzymatic esterification reaction;
      ii) below 2 wt % if a base-catalyzed transesterification follows the enzymatic esterification reaction without further FFA reduction; or
      iii) below 10 wt %, below 5 wt %, such as below 2 wt % if neither neutralization nor base catalyzed transesterification follows the esterification step c).
2. The process of paragraph 1, wherein the product of step (c) (i) is optionally separated into a first light phase L1 (6) comprising esterified oil and FFA below 10 wt %, and a first heavy phase H1 (7) comprising glycerol.
3. The process of paragraph 1 or 2, wherein the first light phase L1 (6) or the reaction mixture of step (c) (i) is neutralized (2), preferably by alcoholic neutralization, and with optional further addition of alcohol and/or base, resulting in an FFA level below 2 wt %, particularly below 0.5 wt %, more particularly below 0.25 wt %, such as below 0.1 wt %.
4. The process of paragraphs 1-3, wherein neutralization is followed by separation into a second light phase L2 (14) comprising neutralized oil and FFA, and a second heavy phase H2 (8) comprising neutralized glycerol.
5. The process of paragraph 4, wherein the neutralized oil in the second light phase L2 (14) is reacted in a base catalyzed transesterification reaction (3) in presence of alcohol resulting in a biodiesel reaction mixture comprising alkyl ester.
6. The process of paragraph 1, wherein the product of step (c) (ii) is separated into a first light phase L1 (6) comprising esterified oil and FFA below 2 wt %, and a first heavy phase H1 (7) comprising glycerol.
7. The process of paragraph 6, wherein the esterified oil in the first light phase L1 (6) is reacted in a base catalyzed transesterification reaction (3) in presence of alcohol resulting in a biodiesel reaction mixture comprising alkyl ester.
8. The process of paragraphs 5 or 7, wherein the biodiesel reaction mixture comprising alkyl ester is separated into a third light phase L3 (18) comprising a crude biodiesel/alkyl ester product, and a third heavy phase H3 (13) comprising alkaline crude glycerol.
9. The process of paragraphs 5 and 8, wherein the third heavy phase H3 (13) is recycled back to the neutralization step (2) of paragraph 3.
10. The process of paragraph 1, wherein the reaction mixture of step (c) (ii) is reacted in a base catalyzed transesterification reaction (3) in presence of alcohol resulting in a biodiesel reaction mixture comprising alkyl ester.
11. The process of any of the preceding paragraphs, wherein the second heavy phase H2 (8) and/or third heavy phase H3 (13) is recycled back to step (b) of paragraph 1.
12. The process according to any of the preceding paragraphs, comprising acidulation of first heavy phase and further separation of the acidulated first heavy phase into oil and crude glycerin and recycling to the reaction mixture in step (b).
13. The process according to any of the preceding paragraphs, wherein the alcoholic neutralization step is not included, comprising adding acid to neutralize the second heavy phase H2 (8) or third heavy phase H3 (13) before or during recycling to step (b) of paragraph 1.
14. The process of any of the preceding paragraphs, comprising drying of the mixture in step (b) prior to reaction in step (c).
15. The process of paragraph 14, wherein drying comprises separating the heavy phase from the reaction mixture, drying the heavy phase, and adding some or all back into the reaction mixture.
16. The process of any of the preceding paragraphs, wherein the alcohol is a C1-C5 alcohol.
17. The process according to paragraph 16, wherein the alcohol is selected from a group consisting of methanol, ethanol, propanol, butanol or mixtures thereof.
18. The process according to paragraph 17, wherein the alcohol is methanol or ethanol, particularly methanol.
19. The process according to any of the preceding paragraphs, wherein the process is performed at temperatures in the range of 20-90° C., such as 25-85° C., particularly 30-80° C.
20. The process according to any one of the preceding paragraphs, wherein the substrate is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, including tallow from pigs, beef and sheep, lard, chicken fat, fish oil, palm oil free fatty acid distillate, soy oil free fatty acid distillate, any other fatty acid distillates, soap stock fatty acid material, yellow grease, used cooking oil, palm oil mill effluent and brown grease, partially converted biodiesel, or feedstocks holding some amount of alkyl esters, e.g. FAME, or be part of a blend of feedstocks, such as the olein phase from neutralized biodiesel soapstock, or any combination thereof.
21. The process according to any one of the preceding paragraphs, wherein the esterase is selected from the group consisting of: Aspergillus lipase; Aspergillus niger lipase; Thermomyces lanuginosa lipase; Candida antarctica lipase A; Candida antarctica lipase B; Candida cylindracae lipase; Candida deformans lipase; Candida lipolytica lipase; Candida parapsilosis lipase; Mucor miehei, Chromobacterium; Candida rugosa lipase; Corynebacterium acnes lipase; Humicola lanuginose, Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas sp, ATCC 21808, Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof.
22. The process according to any one of the preceding paragraphs, wherein the esterase is an esterase of SEQ ID NO: 1, SEQ ID NO: 2, SEQ ID NO: 3, SEQ ID NO: 4 or and esterase having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 1, SEQ ID NO: 2, SEQ ID NO: 3 or SEQ ID NO: 4.
23. The process according to any of the preceding paragraphs, wherein the second heavy phase H2 (14) or third heavy phase H3 (18) recycled back to the reaction mixture of step (b) comprises at least 2% wt/wt of the fatty acid feedstock.
24. The process according to any of the preceding paragraphs, wherein the process is continuous, semi-batch, fed batch, or batch.
25. The process according to any of the preceding paragraphs, wherein the esterase in step (b) is not immobilized.
26. The process according to anyone of the preceding paragraphs, wherein the total amount of esterase is within the range of 5-8000 ppm (wt enzyme protein/wt of substrate).
27. The process according to any of the preceding paragraphs, wherein the total amount of said non-immobilized esterase is within the range of 5-1000 ppm (wt enzyme protein/wt of substrate).
28. The process according to paragraphs 25-27, wherein the esterase is added as a liquid, granule, and/or powder.
29. The process according to any of the preceding paragraphs, wherein the FFA content in step (c) (i) is reduced to less than 6 wt %, 4 wt %, 2 wt %, 1.5 wt %, such as less than 1.25 wt %.
30. The process according to any of the preceding paragraphs, wherein the FFA content in step (c) (i) is reduced to the range of 0.5-8 wt %, 0.75-4 wt %, such as 1-2 wt %.
31. The process according to any of the preceding paragraphs, wherein the FFA content in step (c) (ii) is reduced to less than 1.5 wt %, 1.0 wt %, 0.75 wt %, 0.5 wt %, such as less than 0.25 wt %.
32. The process according to any of the preceding paragraphs, wherein the esterase is a TAG-inactive esterase, or is substantially free of TAG-activity.
33. The process of paragraph 1, wherein the reaction time in step (c) is less than 48, 24, 16, 12, 8, 6, 4 hours, such as about 2 hours.
34. The process according to any of the preceding paragraphs, wherein the enzymatic reaction does not result in bound glycerin concentrations below 0.25 wt %, preferably not below 0.5 wt % and most preferably not below 1 wt %.
35. The process according to any of the preceding paragraphs, wherein part or all of the first heavy phase H1 (7) is recycled into the mixture of step (b), or alternatively in case the first separation step is not performed, then part or all of the second heavy phase H2 (8) is recycled into the mixture of step (b).
36. The process according to any of the preceding paragraphs, wherein the FFA concentration in the feedstock does not exceed 90 wt %, 80 wt %, 70 wt %, 50 wt % and particularly 30 wt % or even 15 wt %.
37. The process according to any of the preceding paragraphs, wherein the FFA content in the feedstock is at least 1.5, at least 2, at least 2.5, at least 3, at least 5, such as at least 10 wt %.
38. The process according to any of the preceding paragraphs, wherein the FFA content in the feedstock is in the range from 1-100 wt %, 1-70 wt %, 1-60 wt %, 2-50 wt %, 2-30 wt %, 3-20 wt %, <sz<such as 3-10 wt %.
39. The process according to any of the preceding paragraphs, wherein phospholipase, preferably phospholipase A and/or C are at least one or more of the esterases added in step (b).
40. The process according to paragraph 1 comprising the steps of:
   (a) providing a fatty acid feedstock comprising free fatty acids (FFA) in the range from 2 wt % to 30 wt %;
   (b) contacting said fatty acid feedstock with a composition comprising a TAG-inactive esterase and alcohol and glycerol; and
   (c) incubating the mixture of step b) under conditions allowing the esterase to catalyze esterification of FFA until FFA levels are reduced below 2 wt %; and further comprising
   (d) separating the product of step (c) into a first light phase L1 and a first heavy phase H1; and
   (e) neutralizing the residual FFA in the light phase L1 in an alcoholic neutralization step (2); and
   (f) separation of the neutralization product of step (e) in a second light phase L2 (14) and a second heavy phase H2 (8); and
   (g) base catalyzed transesterification of the light phase L2 (14) to produce a crude biodiesel reaction mixture.
41. The process according to paragraph 40, wherein some or all of the alcohol, particularly methanol, and/or glycerol stems from the alcoholic neutralization step (2).
42. The process according to any of paragraphs 40-41, wherein reaction time of step (c) does not exceed 8 hours.
43. The process according to any of paragraphs 40-42, wherein at least 1%, preferably at least 4% total glycerin remains unconverted after step (c).
44. The process of paragraph 40, wherein the esterase is selected from an esterase having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 1.
45. The process according to paragraph 1 comprising the steps of:
   (a) providing fatty acid feedstock comprising more than 1 wt % Free Fatty Acids (FFA);
   (b) contacting said fatty acid feedstock with a composition comprising an esterase and alcohol and glycerol; and
   (c) incubating the mixture of step (b) under conditions allowing the esterase to catalyze esterification of FFA until FFA levels are reduced below 1 wt %; and in case the amount of water in the incoming streams and FFA in the feedstock results in water concentrations that leads to FFA concentrations exceeding 1 wt % at equilibrium, then at least one of the following steps is applied:
      (c) (i) Drying the reaction mixture, and/or
      (c) (ii) Drying incoming streams of oil, and/or glycerol, and/or
      (c) (iii) Separating the light phase from the heavy phase, drying the heavy phase, and recycling at least part of the dried heavy phase back into step (c);
   (d) separating the product of step (c) into a first light phase L1 (6) and a first heavy phase H1 (7), which optionally is combined with step (c) (iii); and
   (e) base catalyzed transesterification of the second light phase L2 to produce a crude biodiesel reaction mixture.
46. The process according to paragraph 45, wherein the esterase is a TAG-inactive esterase, or is substantially free of TAG-activity.
47. The process according to any of paragraphs 45-46, wherein the esterase is selected from an esterase having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 1.
48. The process according to any of paragraphs 45-47, wherein reaction time of step (c) does not exceed 48 hours, and preferably does not exceed 24 hours, and most preferably does not exceed 12 hours.
49. The process according to any of paragraphs 45-48, wherein at least 5% of the original total glycerin remains unconverted after step (c).
50. The process according to any of paragraphs 45-49, wherein at least 50% of the original total glycerin remains unconverted after step (c).
51. The process according to any of paragraph 45-50, wherein at least the glycerol is provided by neutralization of the third heavy phase (H3).
52. The process according to any of paragraphs 45-51, wherein the total amount of water present is kept below 1 wt % of the total reaction mixture mass.
53. A process for producing a crude biodiesel mixture, said process comprising an enzymatic pretreatment according to paragraph 1, and further comprising at least one separation step of a light and a heavy phase, and comprising at least a base catalyzed transesterification step to produce the crude biodiesel mixture comprising alkyl ester.
54. An enzymatic process for producing an oil suitable as fuel for ships and/or as an oil feedstock for further downstream processing not involving transesterification, comprising the steps of:
   (a) providing fatty acid feedstock comprising more than 1 wt % Free Fatty Acids (FFA);
   (b) contacting said fatty acid feedstock with a composition comprising an esterase and at least one component selected from the group consisting of alcohol, alcohol and glycerol, alcohol and water, alcohol and water and glycerol to form a mixture; and
   (c) incubating the mixture of step (b) under conditions allowing the esterase to catalyze esterification of FFA until FFA levels are reduced below 10 wt %, below 5 wt %, such as below 2 wt %.
55. The process of paragraphs 1 c) iii) or 54, wherein the alcohol is methanol or ethanol, particularly ethanol.
56. The process of paragraphs 54-55, further comprising a hydrotreatment step, yielding aliphatic hydrocarbons.
57. The process according to any of paragraphs 1 c) iii) or 54, wherein the esterase is selected from an esterase having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 1, SEQ ID NO: 2, SEQ ID NO: 3, or SEQ ID NO: 4.
58. The process according to any of paragraphs 1 c) iii) or 54-57, wherein the esterase is a TAG-inactive esterase, or is substantially free of TAG-activity, such as SEQ ID NO: 1.
59. The process of paragraph 58, wherein the esterase is not immobilized, e.g., is present as a liquid formulation, as a granule, and/or as powder.
60. A use of the oil product of paragraphs 1 c) iii) or 54 as a fuel for ships or as a feedstock oil for hydrotreated vegetable oil (HVO) production.

The present invention is further described by the following example that should not be construed as limiting the scope of the invention.

### EXAMPLES

### Example 1: FFA Esterification by Largely Tri-Acyl-Glycerol (TAG)-Inactive Enzyme

Crude palm oil (CPO) as source of feedstock oil comprising 4.4 wt % FFA, 6.1 wt % DAG, 0 wt % FAME, the rest being mainly TAG. Esterase of SEQ ID NO: 1 was used as it shows little reactivity on TAG, which was beneficial as it allows for dosage of methanol stoichiometrically only to the FFA, MAG and DAG. The esterase was dosed as a solution holding about 0.85 wt % active enzyme protein, and the enzyme dosages in the table below are dosages of the solution rather than esterase protein. Methanol was dosed and reported below in equivalents based on total fatty acids in the feedstock oil, with 1 equivalent being roughly 11% (wt/wt of oil) based on an assumed mean molar weight of 279 g/mol of fatty acids in the oil. The oil was melted, and 30 g was weighed off. Then glycerol and methanol were added, and the mixture was pre-heated to 40° C. Finally, the esterase (SEQ ID NO: 1) was dosed at time zero. Reaction was performed in a shaking incubator oven in square 100 mL blue-cap bottles at 250 rpm. In the tables below, the remainder up to 100% is mostly triglyceride and small amounts of MAG, which was not measured here.

Methods for analysis: FFA by AOCS Official Method Ca 5a-40. FAME measured by a customized NMR method. DAG by a customized HPLC method.

TABLE 1

| equiv. | Gylcerol | Esterase (SEQ ID NO: | Time | FFA | FAME | |
|---|---|---|---|---|---|---|
| Methanol | (%) | 1) | (hours) | (wt %) | (wt %) | DAG (wt %) |
| 0.1 | 0 | 0.1% | 2 | 1.58 | 4.7 | 4.78 |
| | | | 24 | 1.11 | 4.5 | 3.27 |
| | | 0.46% | 2 | 1.68 | 4.82 | 3.34 |
| | | | 24 | 1.8 | 7.68 | 2.13 |
| | 5 | 0.1% | 2 | 1.78 | 3.91 | 3.64 |
| | | | 24 | 1.51 | 7.64 | 2.51 |
| | | 0.46% | 2 | 0.58 | 6.42 | 2.88 |
| | | | 24 | 2.81 | 8.09 | 2.74 |
| | 10 | 0.1% | 2 | 0.89 | 5.78 | 2.95 |
| | | | 24 | 1.45 | 7.93 | 2.33 |
| | | 0.46% | 2 | 0.47 | 7.07 | 1.25 |
| | | | 24 | 2.57 | 7.99 | 2.8 |
| 0.2 | 0 | 0.1% | 2 | 3.07 | 1.02 | 5.58 |
| | | | 24 | 0.66 | 5.39 | 10.55 |
| | | 0.46% | 2 | 1.12 | 3.58 | 4.07 |
| | | | 24 | 0.46 | 7.59 | 2.99 |
| | 5 | 0.1% | 2 | 0.95 | 5.92 | 2.86 |
| | | | 24 | 0.52 | 8.27 | 2.61 |
| | | 0.46% | 2 | 0.27 | 7.42 | 2.26 |
| | | | 24 | 1.34 | 10.16 | 2.26 |
| | 10 | 0.1% | 2 | 0.88 | 6.08 | 2.91 |
| | | | 24 | 0.51 | 8.52 | 1.74 |
| | | 0.46% | 2 | 0.23 | 7.75 | 2.45 |
| | | | 24 | 1.74 | 12.22 | 2.64 |
| 0.4 | 0 | 0.1% | 2 | 3.73 | 0.16 | 5.52 |
| | | | 24 | 3.71 | 0.59 | 6.96 |
| | | 0.46% | 2 | 2.85 | 1.64 | 5.14 |
| | | | 24 | 1.51 | 4.27 | 35.80 |
| | 5 | 0.1% | 2 | 2.13 | 3.47 | 4.15 |
| | | | 24 | 0.2 | 8.47 | 4.56 |
| | | 0.46% | 2 | 0.33 | 7.06 | 3.12 |
| | | | 24 | 0.42 | 9.17 | 3.5 |
| | 10 | 0.1% | 2 | 1.22 | 5.54 | 3.31 |
| | | | 24 | 0.29 | 9.48 | 3.86 |
| | | 0.46% | 2 | 0.19 | 7.67 | 2.96 |
| | | | 24 | 0.58 | 11.36 | 3.7 |
| 1 | 0 | 0.1% | 2 | 3.62 | 0.62 | 5.71 |
| | | | 24 | 3.55 | 0.73 | 6.82 |
| | | 0.46% | 2 | 3.21 | 1.12 | 5.56 |
| | | | 24 | 2.47 | 2.69 | 6.29 |
| | 5 | 0.1% | 2 | 3.72 | 0.56 | 5.22 |
| | | | 24 | 3.61 | 0.01 | 5.98 |
| | | 0.46% | 2 | 3.46 | 0.97 | 5.51 |
| | | | 24 | 3.22 | 1.49 | 5.74 |
| | 10 | 0.1% | 2 | 3.58 | 0.8 | 5.69 |
| | | | 24 | 1.85 | 4.78 | 5.83 |
| | | 0.46% | 2 | 2.72 | 2.66 | 5 |
| | | | 24 | 3.44 | 1.34 | 5.46 |

From Table 1, FFA levels are well below 0.5% after just 2 hrs reaction in many cases, and even below 0.25% in some. After 2 hrs reaction time, TAG was largely unconverted. But with 24 hrs reaction time, esterase of SEQ ID NO:1 does convert up to 7% of the TAG at certain conditions, based on the sum of measured components in the tables. The reaction time was significantly reduced compared to enzymatic pre-treatment in the art using alternative lipases, which was primarily the result of use of the largely TAG-inactive enzyme. By not converting TAG, methanol was only consumed in conversion of primarily FFA, the main component of interest, and MAG and DAG. This means much less methanol is required stoichiometrically to reduce FFA compared to known methods in the art. It also means that most of the enzyme activity is occupied in converting a relevant component, rather than in conversion of TAG, which was not important at the stage in the biodiesel production process, where this invention is intended to operate.

The example further shows that there is an optimum in methanol and glycerol addition, where reaction is optimally fast, with optimally low FFA, at an early timepoint in the extent of reaction. At low methanol dosages, there is simply not enough methanol to affect efficient and quick conversion, and at high methanol dosages, the enzyme stability suffers. All enzymes known to exhibit at least some decrease in stability in presence of methanol. More glycerol was generally preferable, because it dilutes the methanol, resulting in increased enzyme stability, and it reduces the activity of water, affecting lower FFA levels at equilibrium. More enzyme is generally better but comes at an economical cost.

### Example 2: FFA Esterification Comparison of TAG-Active and Largely TAG-Inactive Enzyme-1

The feedstock oil used was prepared as a 1 L stock mixture in a ratio of 30 crude palm oil (CPO) to 1 oleic acid (technical grade, >90 wt % oleic acid, Sigma Aldrich). The stock feedstock comprised 8.49 wt % FFA and 0 wt % FAME, the rest being mainly MAG, DAG and TAG. Esterase of SEQ ID NO:1 or SEQ ID NO:2 was employed. The enzymes were dosed as solutions holding about 0.85 wt % and 3.6 wt % active enzyme protein, respectively, and the enzymes were dosed in an amount of 0.1% (wt of enzyme solution/wt of oil). Methanol, water, and glycerol were dosed in % (wt/wt of oil). The feedstock was melted at 60° C., and 30 g was weighed off. 20 ppm NaOH was added as 2N solution. Then glycerol, water and methanol were added, and the mixture was pre-conditioned to 35° C. Finally, the enzyme was dosed at time zero. Reaction ran in shaking incubator oven in square 100 mL blue-cap bottles at 250 rpm.
5 In the results table below, the remainder up to 100% is mostly MAG, DAG and TAG, which were not measured.

Methods for analysis: FFA by AOCS Official Method Ca 5a-40. FAME measured by a customized NMR method.

TABLE 2

| | Methanol | Water | Glycerol | Time | FFA | FAME |
|---|---|---|---|---|---|---|
| Esterase | (%) | (%) | (%) | (hour's) | [%] | [%] |
| Feedstock CPO + 3.3% (1 g/30 | | | | | 8.49 | 0 |
| g oil) Oleic Acid added | | | | | | |
| SEQ ID | 2 | 0.5 | 0 | 1 | 7.67 | 1.4 |
| NO: 1 | | | | 3 | 5.9 | 3.7 |
| | | | | 4 | 4.84 | 5.2 |
| | | | | 6 | 3.51 | 7.4 |
| | | | | 24 | 0.8 | 10.7 |
| | | 1 | 0 | 1 | 7.52 | 1.6 |
| | | | | 3 | 5.56 | 4.2 |
| | | | | 4 | 4.6 | 5.5 |
| | | | | 6 | 3.42 | 7.4 |
| | | | | 24 | 0.83 | 10.6 |
| | | 0 | 5 | 1 | 7 | 2.2 |
| | | | | 3 | 4.07 | 5.6 |
| | | | | 4 | 2.54 | 8.1 |
| | | | | 6 | 0.99 | 9.8 |
| | | | | 24 | 0.35 | 10.6 |
| | 4 | 0.5 | 0 | 1 | 7.61 | 1.4 |
| | | | | 3 | 6.79 | 2.3 |
| | | | | 4 | 6.16 | 3.2 |
| | | | | 6 | 5.61 | 5.7 |
| | | | | 24 | 2.24 | 8.4 |
| | | 1 | 0 | 1 | 7.57 | 1.5 |
| | | | | 3 | 6.35 | 2.6 |
| | | | | 4 | 5.84 | 3.5 |
| | | | | 6 | 5.29 | 5.5 |
| | | | | 24 | 1.77 | 9 |
| | | 0 | 5 | 1 | 6.7 | 2.6 |
| | | | | 3 | 4.56 | 5 |
| | | | | 4 | 3.68 | 6.1 |
| | | | | 6 | 2.59 | 8.3 |
| | | | | 24 | 0.22 | 11 |
| | 6 | 0.5 | 0 | 1 | 7.75 | 1.3 |
| | | | | 3 | 7.2 | 1.4 |
| | | | | 4 | 6.91 | 2.2 |
| | | | | 6 | 6.38 | 6.5 |
| | | | | 24 | 3.52 | 6.6 |
| | | 1 | 0 | 1 | 7.4 | 1.5 |
| | | | | 3 | 6.49 | 2.1 |
| | | | | 4 | 6.32 | 3.1 |
| | | | | 6 | 5.63 | 6.8 |
| | | | | 24 | 2.05 | 8.6 |
| | | 0 | 5 | 1 | 7.78 | 1.8 |
| | | | | 3 | 6.83 | 2.7 |
| | | | | 4 | 6.35 | 3.6 |
| | | | | 6 | 5.53 | 7.2 |
| | | | | 24 | 1.8 | 9.4 |
| SEQ ID | 2 | 0.5 | 0 | 1 | 10.65 | 10.4 |
| NO: 2 | | | | 3 | 12.34 | 14 |
| | | | | 4 | 12.76 | 14.5 |
| | | | | 6 | 12.84 | 14.6 |
| | | | | 24 | 13.96 | 15.3 |
| | | 1 | 0 | 1 | 11.62 | 8.6 |
| | | | | 3 | 14.34 | 14.2 |
| | | | | 4 | 15.27 | 14.4 |
| | | | | 6 | 15.65 | 14.9 |
| | | | | 24 | 17.1 | 15.4 |
| | | 0 | 5 | 1 | 4.32 | 7.7 |
| | | | | 3 | 5.25 | 15.1 |
| | | | | 4 | 5.06 | 15.4 |
| | | | | 6 | 5.91 | 16 |
| | | | | 24 | 6.44 | 16.5 |
| | 4 | 0.5 | 0 | 1 | 8.1 | 18.9 |
| | | | | 3 | 9.1 | 27.5 |
| | | | | 4 | 9.23 | 28.8 |
| | | | | 6 | 9.31 | 29.1 |
| | | | | 24 | 10.26 | 30.1 |
| | | 1 | 0 | 1 | 10.35 | 21.7 |
| | | | | 3 | 11.21 | 28.6 |
| | | | | 4 | 11.49 | 29.9 |
| | | | | 6 | 11.36 | 30.1 |
| | | | | 24 | 13.63 | 31.6 |
| | | 0 | 5 | 1 | 7.77 | 2.6 |
| | | | | 3 | 7.42 | 5.5 |
| | | | | 4 | 7.17 | 7.6 |
| | | | | 6 | 6.71 | 12.3 |
| | | | | 24 | 4.62 | 26.3 |
| | 6 | | | 1 | 6.25 | 20.1 |
| | | 0.5 | | 3 | 6.35 | 37.2 |
| | | | 0 | 4 | 6.4 | 39.1 |
| | | | | 6 | 6.39 | 43.2 |
| | | | | 24 | 7.36 | 46 |
| | | 1 | | 1 | 7.95 | 26.5 |
| | | | | 3 | 8.18 | 38.8 |
| | | | 0 | 4 | 8.09 | 40.6 |
| | | | | 6 | 7.77 | 42 |
| | | | | 24 | 9.45 | 45.6 |
| | | | | 1 | 8.07 | 1.3 |
| | | 0 | 5 | 3 | 8.05 | 1 |
| | | | | 4 | 7.75 | 12.9 |
| | | | | 6 | 7.47 | 5.0 |
| | | | | 24 | 7.87 | 1.3 |

### Example 3: FFA Esterification Comparison of TAG-Active and Largely TAG-Inactive Enzyme-2

Example 2 above indicated that too much methanol might have been added, exceeding the methanol tolerance of especially SEQ ID NO:1. This example is largely similar to example 2, but examines lower methanol dosages, higher glycerol dosages and includes combinations of water and glycerol additions.

The feedstock oil used was prepared as a 1 L stock mixture in a ratio of 30 crude palm oil (CPO) to 1 oleic acid (technical grade, >90 wt % oleic acid, Sigma Aldrich). The stock feedstock comprised 8.49 wt % FFA and 0 wt % FAME, the rest being mainly MAG, DAG and TAG. Esterase of SEQ ID NO: 1 or SEQ ID NO:2 enzymes were employed. The enzymes were dosed as solutions holding about 0.9 wt % and 3.6 wt % active enzyme protein, respectively, and the enzymes were dosed in an amount of 0.1% (wt of enzyme solution/wt of oil). Methanol, water, and glycerol were dosed in % (wt/wt of oil). The feedstock was melted at 60° C., and 30 g was weighed off. 50 ppm NaOH was added as 2N solution. Then glycerol, water and methanol were added, and the mixture was pre-conditioned to 35° C. Finally, the enzyme was dosed at time zero. Reaction ran in shaking incubator oven in square 100 mL blue-cap bottles at 250 rpm. In the results table below, the remainder up to 100% is mostly MAG, DAG and TAG, which were not measured.

Methods for analysis: FFA by AOCS Official Method Ca 5a-40. FAME measured by a customized NMR method.

TABLE 3

| | | | | FFA | FAME |
|---|---|---|---|---|---|
| | | | | [%] | [%] |
| Feedstock CPO + 3.3% (1 g/30 g oil) Oleic Acid | | | | 7.8 | 0.0 |
| added | | | | | |
| SEQ ID | 10% | 0.15% Water | 1 h | 5.9 | 2.2 |
| NO: 1 | Glycerol | 1.87% | 3 h | 3.0 | 6.0 |
| | | Methanol | 6 h | 0.6 | 9.9 |
| | | | 24 h | 0.39 | 10.8 |
| | | 0% Water | 1 h | 6.5 | 1.3 |
| | | 1% Methanol | 3 h | 2.9 | 4.2 |
| | | | 6 h | 1.8 | 7.5 |
| | | | 24 h | 1.47 | 7.5 |
| | | 0.5% Water | 1 h | 5.7 | 2.3 |
| | | 2% Methanol | 3 h | 3.0 | 6.1 |
| | | | 6 h | 0.7 | 10.0 |
| | | | 24 h | 0.40 | 10.7 |
| | | 1% Water | 1 h | 6.4 | 2.4 |
| | | 2% Methanol | 3 h | 2.7 | 6.7 |
| | | | 6 h | 0.7 | 9.8 |
| | | | 24 h | 0.50 | 10.7 |
| | 0% | 0.5% Water | 1 h | 7.5 | 0.3 |
| | Glycerol | 1% Methanol | 3 h | 7.3 | 0.7 |
| | | | 6 h | 6.6 | 1.9 |
| | | | 24 h | 6.68 | 3.8 |
| | | 0.5% Water | 1 h | 7.3 | 0.3 |
| | | 2% Methanol | 3 h | 7.2 | 0.7 |
| | | | 6 h | 6.8 | 1.7 |
| | | | 24 h | 6.70 | 3.8 |
| | | 1% Water | 1 h | 7.2 | 0.5 |
| | | 1% Methanol | 3 h | 7.0 | 1.2 |
| | | | 6 h | 5.8 | 3.1 |
| | | | 24 h | 2.91 | 7.2 |
| | | 1% Water | 1 h | 7.3 | 0.8 |
| | | 2% Methanol | 3 h | 6.6 | 1.8 |
| | | | 6 h | 5.4 | 3.3 |
| | | | 24 h | 1.98 | 8.2 |
| SEQ ID | 10% | 0.15% Water | 1 h | 5.4 | 8.0 |
| NO: 2 | Glycerol | 1.87% | 3 h | 3.9 | 14.2 |
| | | Methanol | 6 h | 3.9 | 15.7 |
| | | | 24 h | 5.00 | 16.5 |
| | | 0% Water | 1 h | 6.6 | 2.6 |
| | | 1% Methanol | 3 h | 7.4 | 7.1 |
| | | | 6 h | 3.8 | 8.6 |
| | | | 24 h | 4.53 | 8.0 |
| | | 0.5% Water | 1 h | 5.1 | 9.5 |
| | | 2% Methanol | 3 h | 4.7 | 14.8 |
| | | | 6 h | 5.3 | 15.8 |
| | | | 24 h | 5.08 | 16.2 |
| | | 1% Water | 1 h | 5.5 | 8.9 |
| | | 2% Methanol | 3 h | 6.1 | 14.5 |
| | | | 6 h | 7.3 | 15.5 |
| | | | 24 h | 7.89 | 16.9 |
| | 0% | 0.5% Water | 1 h | 11.5 | 2.2 |
| | Glycerol | 1% Methanol | 3 h | 13.7 | 3.2 |
| | | | 6 h | 15.7 | 3.9 |
| | | | 24 h | 17.08 | 3.9 |
| | | 0.5% Water | 1 h | 11.0 | 1.9 |
| | | 2% Methanol | 3 h | 13.8 | 3.2 |
| | | | 6 h | 15.5 | 3.9 |
| | | | 24 h | 16.02 | 3.9 |
| | | 1% Water | 1 h | 14.4 | 5.6 |
| | | 1% Methanol | 3 h | 17.5 | 7.4 |
| | | | 6 h | 18.8 | 7.4 |
| | | | 24 h | 21.13 | 7.6 |
| | | 1% Water | 1 h | 12.9 | 12.3 |
| | | 2% Methanol | 3 h | 15.0 | 15.0 |
| | | | 6 h | 14.3 | 15.1 |
| | | | 24 h | 17.83 | 15.7 |

From Example 2 and 3, it is seen that employment of a TAG-inactive (SEQ ID NO: 1) enzyme within the scope of the invention is most preferable, because it allows for low and fast dosage of methanol and subsequent quick reaction at relatively low enzyme dosage, especially when taking into account the relative amounts of active enzyme 5 protein dosed.

Still, when employing the TAG-active SEQ ID NO: 2, a reduction in FFA levels is achievable, within the parameters of this examples. Such FFA reduction is only observed in cases where glycerol is added and preferably without significant addition 10 of water. This illustrates the most important technical basis of the invention, namely the significance of the chemical equilibrium between FFA+methanol and FAME+water. This equilibrium is critically dependent on the amount of methanol relative to water. And because of methanol being consumed in what is de facto irreversible conversion of TAG to FAME, this equilibrium will shift to increasing FFA levels, as methanol is converted through TAG conversion, and as FAME and/or water is increased accordingly. Thus, keeping water levels low and methanol dosage high, is more important with a TAG-active enzyme than with a TAG-inactive enzyme. Presence of glycerol is known to reduce water activity, resulting in a reduction of achievable FFA concentrations at equilibrium. Further, glycerol enables reaction without addition of water, while no addition of either water or glycerol is known to result in little to no reaction due to the requirement for a heavy phase. Therefore, in order to reduce addition of water, addition of glycerol is advisable. Employment of a TAG-active enzyme in the invention is not impossible, though, as cited prior arts show that reduction of FFA to the target levels is still possible. One main difference between this example and the state-of-the-art way of employment of SEQ ID NO: 2 is especially the dosage and dosing rate of methanol. In the enzymatic transesterification reaction, with a target of obtaining finished biodiesel rather than a pretreatment as in the present invention, methanol dosages typically exceed 15% (wt/wt of oil). However, the dosing rate of methanol is relatively slow, being typically linear over 10 hours or more in batch reactions as an example, allowing for gradual consumption of the methanol before levels critical to the enzyme stability are reached. In the final stages of reaction, where the total dosage of methanol exceeds what can stoichiometrically be converted, there is accumulation of methanol and a resulting chemical equilibrium that pushes FFA down to levels as low as 1 wt %, simply because there is little or no more MAG, DAG and TAG that can be converted through consumption of methanol. Therefore, employment of a TAG-active enzyme in the present invention preferably involves a high and gradual dosage of methanol to make up for methanol, which is lost to unnecessary conversion of especially TAG. Reaction time, enzyme dosage, choice of water and/or glycerol dosage must then be adjusted accordingly to arrive at a meaningful design based on the invention. For example, the targets of the invention would be achievable using a relatively high dosage of TAG-active enzyme, a relatively high but preferably gradual dosage of methanol in presence of an amount of glycerol and/or water that allows for sufficient enzyme stability while still reaching target FFA levels. This would enable achieving FFA<2 wt % within the full scope of the invention, while optionally leaving considerable amounts of unconverted glycerides that must be converted in a further chemical transesterification reaction. The expert in the field would recognize such ways of employing TAG-active enzymes within the scope of the invention. Such use of TAG-active enzymes is preferable only in cases where such enzymes can be obtained at significantly lower cost than TAG-inactive enzymes, thus enabling reaction rates that are competitive with those achievable using TAG-inactive enzymes through increased enzyme dosage.

### Example 4: Dosage Response of Esterase of SEQ ID NO:1 at Two Methanol Dosages in Glycerol and Water

The feedstock oil used was prepared as a 1 L stock mixture in a ratio of 30 crude palm oil (CPO) to 1 oleic acid (technical grade, >90 wt % oleic acid, Sigma Aldrich). The stock feedstock comprised 8.49 wt % FFA and 0 wt % FAME, the rest being mainly MAG, DAG and TAG. The enzyme was dosed as a solution holding about 0.9 wt % active enzyme protein, and the enzyme was dosed in variable amounts as indicated in the results table in % (wt of enzyme solution/wt of oil). Methanol as indicated in the results table, 0.5% water, and 10% glycerol were all dosed in % (wt/wt of oil). The feedstock was melted at 60° C., and 60 g was weighed off. 50 ppm NaOH was added as 2N solution. Then glycerol, water and methanol were added, and the mixtures were pre-conditioned to 45° C. Finally, the enzyme was dosed at time zero. Reaction ran in magnetic stirred round 100 mL blue-cap bottles in water batch at 700 rpm. In the results table below, the remainder up to 100% is mostly MAG, DAG and TAG, which were not measured.

Methods for analysis: FFA by AOCS Official Method Ca 5a-40. FAME measured by a customized NMR method.

TABLE 4

| | | | FFA | FAME |
|---|---|---|---|---|
| | | | [%] | [%] |
| Feedstock CPO + 3.3% Oleic Acid | | | 8.56 | 0 |
| added | | | | |
| 0.05% SEQ ID | 2% Methanol | 1 h | 5.61 | 2.5 |
| NO: 1 | | 2 h | 3.90 | 5.1 |
| | | 3 h | 2.09 | 6.8 |
| | | 4 h | 1.76 | 7.6 |
| | | 6 h | 0.84 | 9.3 |
| | | 24 h | 0.52 | 10 |
| | 4% Methanol | 1 h | 6.17 | 2.6 |
| | | 2 h | 4.71 | 5 |
| | | 3 h | 3.53 | 6.6 |
| | | 4 h | 2.40 | 7.9 |
| | | 6 h | 1.29 | 9.5 |
| | | 24 h | 0.21 | 11.3 |
| 0.1% SEQ ID NO: 1 | \|2% Methanol | 1 h | 4.83 | 2.8 |
| | | 2 h | 2.75 | 6.2 |
| | | 3 h | 1.74 | 8.2 |
| | | 4 h | 0.92 | 8.7 |
| | | 6 h | 0.50 | 10.2 |
| | | 24 h | 0.60 | 10.1 |
| | 4% Methanol | 1 h | 5.35 | 3.8 |
| | | 2 h | 3.32 | 6.4 |
| | | 3 h | 2.17 | 8 |
| | | 4 h | 1.22 | 9.3 |
| | | 6 h | 0.60 | 10.3 |
| | | 24 h | 0.35 | 10.5 |
| 0.2% SEQ ID NO: 1 | 2% Methanol | 1 h | 2.56 | 4.8 |
| | | 2 h | 1.14 | 7.9 |
| | | 3 h | 0.61 | 9.3 |
| | | 4 h | 0.49 | 9.2 |
| | | 6 h | 0.48 | 9.5 |
| | | 24 h | 0.92 | 9.5 |
| | 4% Methanol | 1 h | 1.92 | 8.2 |
| | | 2 h | 0.54 | 10.2 |
| | | 3 h | 0.27 | 10.6 |
| | | 4 h | 0.21 | 10.2 |
| | | 6 h | 0.22 | 10.9 |
| | | 24 h | 0.19 | 11.71 |

The results show a dosage response of SEQ ID NO:1 enzyme. Further, temperature is notable increased from 35° C. to 45° C. relative to the prior examples and mixing improved by moving to 700 rpm magnetic stirring rather than 250 rpm shaking. 10% glycerol and 0.5% water is dosed in all cases based on the results of example 3, balancing the negative impact of water on achievable FFA levels at equilibrium and the positive impact of water on the enzyme stability. These improvements result in an increased rate of reaction. As the least possible amount of enzyme is preferably employed for economical reason, a mere 3-4 hours of reaction with 0.05% SEQ ID NO: 1 and 2% methanol was sufficient to arrive at less than 2 wt % FFA. Further, it is even possible to arrive at FFA concentrations resembling those achievable when employing deodorization, neutralization, or other efficient means of FFA reduction pretreatments, reaching 0.21 wt % FFA at a timepoint between 6 h and 24 h of reaction when dosing 4% methanol, or even after just 3-4 hours using 0.2% SEQ ID NO: 1. Increasing the dosage of SEQ ID NO: 1 yields significant improvements. As SEQ ID NO: 1 is commercially available and widely used industrially today in other applications, dosages of 0.1 or 0.2% are fair and economically viable. It is further known that SEQ ID NO: 1 is recyclable through recycling of the glycerol or water-based heavy phase after separation, making accumulation and concentration of the enzyme to levels such as 0.1 and 0.2% achievable even with a net dosage of just 0.05% enzyme.

### Example 5: <0.25 wt % FFA is Obtainable Using TAG-Active Enzyme at Optimal Conditions

CPO (4.4 wt % FFA, 6.1 wt % DAG) oil was dried in a rotary evaporator at 80° C. under 5 mbar vacuum for 1 hour. 30 g pre-dried CPO was then weighed off in 100 mL square bottle followed by addition of 20% (wt/wt of oil) dry technical grade glycerol. The mixture was conditioned in a shaking incubator oven at 35° C. under 250 rpm mixing for 30 minutes to ensure that the temperature was reached. 100 ppm NaOH was added as 50% aqueous solution. 0.25 or 0.5% (wt/wt of oil) enzyme of SEQ ID NO: 2 was added at timepoint 0 h. Reaction ran at 35° C. in the shaking incubator at 250 rpm under 40 hrs continuous linear dosing of 2 molar equivalents of methanol based on free and bound fatty acids. After methanol dosing completion at the 40 hours mark, reaction is continued until the 72 hours mark and the results are measured. At the 24 hours mark, only 24/40 of the 2 equivalents of methanol would have been dosed. 2 mL samples are taken by pipette into 2 mL Eppendorf tubes. Samples are incubated for 10 minutes at 99° C. to inactivate the enzyme before being centrifuged at 2000 rpm for 1 minute. Finally, the samples are dried under vacuum at 80° C. for 2 hours to remove methanol.

Methods for analysis: FFA by AOCS Official Method Ca 5a-40. Mono-di, and triglycerides by AOCS Official Method Ck 2-09 using the Quality Trait Analysis (QTA) NIR method offered.

TABLE 5

| Enzyme Dosage | | | | | |
|---|---|---|---|---|---|
| (Lipase of SEQ | Reaction | MG, | DG, | TG, | FFA, |
| No: 2) | Time | wt % | wt % | wt % | wt % |
| 0.25% | 24 h | 3.78 | 3.68 | 5.65 | 0.39 |
| | 48 h | 1.27 | 0.61 | 0.36 | 0.21 |
| | 72 h | 0.7 | 0.18 | 0.06 | 0.24 |
| 0.5% | 24 h | 3.06 | 2.9 | 4.22 | 0.46 |
| | 48 h | 0.67 | 0.11 | 0.05 | 0.23 |
| | 72 h | 0.6 | 0.06 | 0.02 | 0.25 |

Finished biodiesel standards vary, with EN-14214 describing MAG<0.7 wt %, DAG<0.2 wt %, TAG<0.2 wt % and FFA<0.25 wt %, roughly.

The results show EN-14214-compliant glyceride and FFA levels are achieved using the enzyme of SEQ ID NO: 2, thus enabling production of finished biodiesel with no requirement for further base-catalyzed transesterification. This is done in a single reaction step on a standard crude palm oil quality. Using 0.25% enzyme enables acceptable results in 72 hours while doubling of the enzyme dosage enables similar results in 48 hours. This is, however, a very long reaction time, and is not feasible in most industrial plants.

For the purposes of this invention, this example is meant to show that use of a TAG-active enzyme for reduction of FFA to meaningfully low levels is possible within the scope of the invention, although use of SEQ ID NO: 1 is preferred. The 24 hrs measurements above show FFA well within the claimed FFA target ranges, and FFA would likely have been well within those ranges at significantly earlier timepoints. Further, a significant improvement to the state of the art, and to this previously undisclosed possibility of achieving finished biodiesel enzymatically of this example, is that there is no requirement for glyceride conversion, because the base-catalyzed transesterification reaction will finish the biodiesel reaction regarding the glycerides. Therefore, the required total reaction time for obtaining finished biodiesel (per the EN-14214 standard) relative to the results of this example, can be more than halved.

### Example 6: TAG-Active Enzyme on Near-Pure FFA Feedstock

Technical grade oleic acid procured from Sigma Aldric was used. 30 g oleic acid was then weighed off in 100 mL square bottle followed by addition of the water and methanol, with water dosed in % (wt/wt of feedstock oleic acid). The mixture was conditioned in a shaking incubator oven at 35° C. under 250 rpm mixing for 30 minutes to ensure that the temperature was reached. 0.2 or 0.4% (wt/wt of oil) enzyme of SEQ ID NO: 2 was added at timepoint 0 hrs. Reaction ran at 35° C. in the shaking incubator at 250 rpm. 2 mL samples were taken by pipette into 2 mL Eppendorf tubes. Samples are incubated for 10 minutes at 99° C. to inactivate the enzyme before being centrifuged at 2000 rpm for 1 minute. Finally, the samples are dried under vacuum at 80° C. for 2 hours to remove methanol.

Methods for analysis: FFA by AOCS Official Method Ca 5a-40.

TABLE 6

| | | | Time | FFA, wt % |
|---|---|---|---|---|
| Feedstock: Oleic Acid (C18: 1, 282 g/mol) | | | 0 h | ~100.0 |
| 0.2% SEQ ID | 6% Water | 1.5 Eqv | 1 h | 77.6 |
| NO: 2 | | MeOH | 3 h | 38.8 |
| | | | 5 h | 17.3 |
| | | | 24 h | 5.8 |
| | | 2 Eqv MeOH | 1 h | 94.1 |
| | | | 3 h | 90.9 |
| | | | 5 h | 95.7 |
| | | | 24 h | 88.3 |
| | | 3 Eqv MeOH | 1 h | 93.2 |
| | | | 3 h | 85.1 |
| | | | 5 h | 53.3 |
| | | | 24 h | 50.8 |
| | 12% Water | 1.5 Eqv | 1 h | 77.4 |
| | | MeOH | 3 h | 40.3 |
| | | | 5 h | 18.4 |
| | | | 24 h | 6.9 |
| | | 2 Eqv MeOH | 1 h | 75.8 |
| | | | 3 h | 35.4 |
| | | | 5 h | 11.4 |
| | | | 24 h | 4.6 |
| | | 3 Eqv MeOH | 1 h | 76.8 |
| | | | 3 h | 46.9 |
| | | | 5 h | 18.5 |
| | | | 24 h | 3.4 |
| 0.4% SEQ ID | 6% Water | 1.5 Eqv | 1 h | 59.1 |
| NO: 2 | | MeOH | 3 h | 19.3 |
| | | | 5 h | 10.4 |
| | | | 24 h | 5.6 |
| | | 2 Eqv MeOH | 1 h | 71.1 |
| | | | 3 h | 27.0 |
| | | | 5 h | 6.2 |
| | | | 24 h | 3.9 |
| | | 3 Eqv MeOH | 1 h | 88.2 |
| | | | 3 h | 75.4 |
| | | | 5 h | 45.6 |
| | | | 24 h | 29.5 |
| | 12% Water | 1.5 Eqv | 1 h | 56.6 |
| | | MeOH | 3 h | 21.1 |
| | | | 5 h | 11.8 |
| | | | 24 h | 7.2 |
| | | 2 Eqv MeOH | 1 h | 55.0 |
| | | | 3 h | 6.6 |
| | | | 5 h | 6.8 |
| | | | 24 h | 4.1 |
| | | 3 Eqv MeOH | 1 h | 59.8 |
| | | | 3 h | 10.3 |
| | | | 5 h | 4.0 |
| | | | 24 h | 3.5 |

The results show efficient and quick reduction of FFA in a pure FFA feedstock. The enzyme is TAG-active, but that is irrelevant in this case, where no glycerides nor glycerol is present. FFA levels at equilibrium of around 3-4 wt % are achievable, and with enough enzyme, and optimal water/methanol ratios, such levels are achievable well before 24 hours of reaction. Further, the results clearly show the detrimental effect of methanol on enzyme stability, as well as the positive and opposite effect of water through dilution of methanol in the polar heavy phase, where the enzyme resides.

Balancing water and methanol optimally to arrive at optimal rate of reaction while achieving an acceptable equilibrium FFA level is thus critical. The example also exemplifies what may happen when employing the invention in a high-FFA feedstock, which may hold glycerides. Palm oil mill effluent, POME, typically holds 30-70 wt % FFA, with the rest primarily being a mixture of MAG, DAG and TAG. In such a case, FFA at equilibrium would be even lower, because conversion of FFA releases water. It would especially be lower, when employing an esterase with preferential FFA activity relative to glycerides, because the stoichiometrically required amount of methanol would be lower, yielding a reduction in FFA at equilibrium. Therefore, the example proves that the scope of the invention may include high-FFA feedstocks, although the preferred use of the invention is employment of a largely TAG-inactive esterase on feedstocks with high amounts of TAG such as soy, palm, used cooking oil, tallow, and the like. Further, the results above do not show FFA levels within the full scope of the invention. However, the expert in the art, in light of the examples provided in the invention, would realize, that the above example could be improved further, especially industrially, such as through second reaction steps at improved conditions. With improved conditions being primarily reduced concentrations of water, in later stages of the process and employment of glycerol.

### Example 7: Employing Glycerol Directly from Industrial Scale Alcoholic Neutralization

Actual feedstock and crude glycerol samples were supplied by a Brazilian biodiesel industry. The feedstock is a mixture of degummed soybean oil and tallow. The glycerol was collected after the alcoholic neutralization in the transesterification process. The characterization of both is shown in the tables below.

TABLE 7
Feedstock characterization

| Analysis | Value |
|---|---|
| FFA (%) | 4.15 |
| pH | 3.73 |
| Water (%) | 0.042 |
| Volatile (%) | 0.34 |

TABLE 8
Glycerol characterization

| Analysis | Value |
|---|---|
| Volatile (%) | 31.18 |
| Water (%) | 1.27 |
| Methanol (%) | 29.91 |
| Free alkalinity | 0.0078 |
| (meq NaOH/g glycerol) | |
| Combined alkalinity | 0.37 |
| (meq NaOH/g glycerol) | |
| Density (g/mL) | 1.052 |
| pH | 8.25 |

Feedstock was melted at 45° C. 100 g of feedstock was then weighed off in 250 ml balloon with a magnetic stir bar, followed by addition of 12 or 15% (wt/wt of feedstock) glycerol. The mixture was conditioned on a heating and stirring plate at 45° C. under 620 rpm mixing for 10 minutes to ensure that the temperature and homogenization between the raw material was reached. 0.10% or 0.15% (wt/wt of feedstock) of enzyme SEQ ID NO: 1 was added at timepoint 0 h, having 0.85 wt % SEQ ID NO: 1 protein in the enzyme solution. Reaction was performed at 45° C. on a heating and stirring plate at 620 rpm until the reaction had completed 4 hours. 3 mL samples are taken every 1 hour of the reaction for FFA analysis. In addition, the influence of different amounts of methanol on the reaction was evaluated, since large amounts can inactivate partially or totally the enzyme. To get this, the methanol present in the glycerol was removed by distillation until it reached different contents in the glycerol phase.

Methods for analysis: FFA by AOCS Official Method Ca 5a-40.

TABLE 9

| | Industrially | | | | | | |
|---|---|---|---|---|---|---|---|
| | obtained | SEQ ID | | | | | |
| Methanol | glycerol | NO: 1 | | | | | |
| in glycerol | dosage | solution | % FFA after each hour of reaction | | | | |
| (%) | (%) | (%) | 0 h | 1 h | 2 h | 3 h | 4 h |
| 31.18 | 12 | 0.10 | 4.15 | 4.13 | 3.86 | 3.43 | 3.86 |
| | | 0.15 | 4.15 | 4.14 | 3.31 | 3.35 | 3.59 |
| | 15 | 0.10 | 4.15 | 3.99 | 3.55 | 3.49 | 4.07 |
| | | 0.15 | 4.15 | 4.03 | 3.33 | 3.44 | 3.99 |
| 17.06 | 12 | 0.15 | 4.15 | 2.59 | 1.96 | 1.34 | 1.16 |
| | 15 | | 4.15 | 3.10 | 2.04 | 1.69 | 1.40 |
| 14.37 | 12 | 0.15 | 4.15 | 2.44 | 1.91 | 1.34 | 1.08 |
| | 15 | | 4.15 | 2.39 | 1.63 | 1.70 | 1.05 |
| 10.89 | 12 | 0.10 | 4.15 | 2.66 | 1.92 | 1.49 | 1.26 |
| | | 0.15 | 4.15 | 2.15 | 1.59 | 1.05 | 0.94 |
| | 15 | 0.10 | 4.15 | 2.91 | 2.28 | 2.08 | 1.72 |
| | | 0.15 | 4.15 | 2.09 | 1.52 | 0.95 | 0.86 |
| 8.13 | 12 | 0.10 | 4.15 | 2.58 | 1.64 | 1.42 | 1.08 |
| | | 0.15 | 4.15 | 1.93 | 1.44 | 1.05 | 0.95 |
| | 15 | 0.10 | 4.15 | 2.50 | 1.69 | 1.34 | 1.33 |
| | | 0.15 | 4.15 | 1.89 | 1.60 | 0.99 | 1.09 |

The reactions using glycerol with the original amount of methanol (31,18%), clearly did not take place, since the acidity after 4 hours of reaction practically did not decrease, indicating that there was no conversion of FFA to methyl-ester. However, as it was distilled off some of the methanol present in the glycerol, the conversion of FFA to methyl-ester took place very efficiently. Therefore, it is clear that the use of industrial glycerol (collected after alcoholic neutralization of the transesterification line) is efficient for pre-esterification reaction with SEQ ID NO:1 once methanol content in heavy phase is controlled. It is also clear, that use of industrial quality glycerol, stemming directly from alcoholic neutralization, is a viable source of glycerol and methanol.

### Example 8: Dosage Response of Esterase of SEQ ID NO:1 at Two Different Ethanol Dosages in Glycerol and Water

This example is similar to example 4, however, with use of ethanol rather than methanol. The feedstock oil used was prepared as a 1 L stock mixture in a ratio of 30 crude palm oil (CPO) to 1 oleic acid (technical grade, >90 wt % oleic acid, Sigma Aldrich). The stock feedstock comprised 7.9 wt % FFA and 0 wt % FAEE, the rest being mainly MAG, DAG and TAG. The enzyme was dosed as a solution holding about 0.9 wt % active enzyme protein, and the enzyme was dosed in variable amounts as indicated in the results table in % (wt of enzyme solution/wt of oil). Ethanol with 96% purity, was dosed as indicated in the results table, and 0.5% water and 10% glycerol were dosed in all flasks in % (wt/wt of oil). The feedstock blend was melted at 60° C., and 60 g was weighed off. 50 ppm NaOH was added as 2N solution. Then glycerol, water and ethanol were added, and the mixtures were pre-conditioned to 45° C. Finally, the enzyme was dosed at time zero. Reaction ran in magnetic stirred round 100 mL blue-cap bottles in water batch at 700 rpm. In the results table below, the remainder up to 100% is mostly FAEE, MAG, DAG and TAG, which were not measured.

Methods for analysis: FFA by AOCS Official Method Ca 5a-40.

TABLE 10

| | | | FFA [%] |
|---|---|---|---|
| Feedstock CPO + 3.3% Oleic Acid | | 0 h | 7.9 |
| added | | | |
| 0.05% SEQ ID NO: 1 | 2% Ethanol | 1 h | 5.8 |
| | | 2 h | 5.5 |
| | | 3 h | 4.4 |
| | | 4 h | 4.2 |
| | | 6 h | 3.2 |
| | | 24 h | 1.2 |
| | 4% Ethanol | 1 h | 6.0 |
| | | 2 h | 3.9 |
| | | 3 h | 4.5 |
| | | 4 h | 4.0 |
| | | 6 h | 3.0 |
| | | 24 h | 0.5 |
| 0.1% SEQ ID NO: 1 | 2% Ethanol | 1 h | 5.7 |
| | | 2 h | 5.0 |
| | | 3 h | 3.7 |
| | | 4 h | 3.2 |
| | | 6 h | 2.3 |
| | | 24 h | 1.3 |
| | 4% Ethanol | 1 h | 5.3 |
| | | 2 h | 4.6 |
| | | 3 h | 2.9 |
| | | 4 h | 2.4 |
| | | 6 h | 1.2 |
| | | 24 h | 0.4 |
| 0.2% SEQ ID NO: 1 | 2% Ethanol | 1 h | 3.5 |
| | | 2 h | 2.5 |
| | | 3 h | 1.6 |
| | | 4 h | 1.4 |
| | | 6 h | 1.0 |
| | | 24 h | 1.6 |
| | 4% Ethanol | 1 h | 4.1 |
| | | 2 h | 3.0 |
| | | 3 h | 1.5 |
| | | 4 h | 1.1 |
| | | 6 h | 0.5 |
| | | 24 h | 0.4 |

The results show a dosage response of SEQ ID NO: 1 enzyme with use of ethanol rather than methanol. Notably, the resulting ethyl ester was not measured due to lack of equipment with relevant accuracy at the time of running the trial. The reductions in FFA concentrations and their clear relationship and trend with ethanol dosage proves that the reaction is running with formation of FAEE. This example, when compared to example 4, shows that ethanol reacts more slowly and yields a worse equilibrium, even when accounting for the significant difference in molar mass of methanol and ethanol, which results in different molar dosages when comparing this example and that of example 4. The elevated FFA concentration at equilibrium may at least partially be due to the water present in the impure ethanol used in this example. This example shows the feasible use of ethanol as alcohol within the scope of the invention and proves the feasibility in reducing FFA levels in a feedstock otherwise relevant outside the scope of standard biodiesel production, e.g., in fuels for ships or further technical uses such as hydrated vegetable oils (HVO) production.

### Example 9: Results from 7-10 Ton Per Hour Industrial Trial

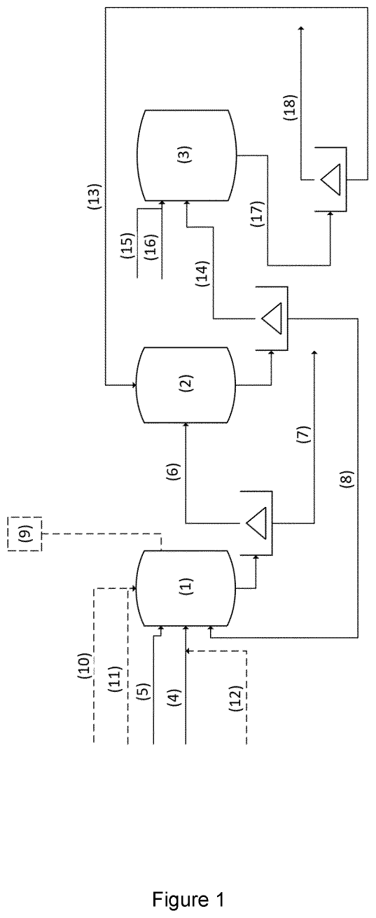

An industrial trial was run at full industrial scale by implementing the findings of the prior examples and the process as described above and in FIG. 1 in an existing facility originally operating a sequence of acid-esterification followed by alcoholic neutralization and then final chemical transesterification.

The existing facility operated using three CSTR reactors in series, and at 7 ton/hr flowrate the total available reaction time was around 7 h 15 min. At 10 ton/hr flowrate the total reaction time was around 5 hours. At 7000 ton/hr flowrate, residence times were: R1 2.30 hr, R2 3.00 hr, R3 1.30 hr.

To implement the enzymatic process of the invention, a new line rerouting the neutralized glycerol phase (FIG. 1, H2 stream) was implemented. Additionally, enzyme dosage and water dosage lines were implemented for the trial. The existing 3 CSTRs originally used for acid esterification, were directly used as enzymatic reactors utilizing the enzymatic process of the invention.

The feedstock oil used was a mixture of animal fats (9.3-9.8 wt % FFA). Acid oil from soap splitting of the crude glycerol effluent (FIGS. 1, H1) (36.7-46.3 wt % FFA). The animal fats alone as well as blends of the two were tested, with blends around 90 animal fats to 10 acid oil by mass (12.4-13.2 wt % FFA in the blend). Glycerol dosages to the reactor were 11-16% (wt/wt oil feed stream), and the glycerol comprised 14-23 wt % methanol during the trial, with reduced methanol concentrations later in the trial due to optimizations. Additional methanol dosage was an available option by existing lines. The enzyme was dosed as a solution holding around 0.9 wt % active enzyme protein, and the enzyme was dosed in an amount of 0.1-0.17% (wt/wt of oil feed stream). The reaction temperature was 43-45 C. Mixing was done by agitation in each vessel.

Methods for analysis: FFA by AOCS Official Method Ca 5a-40.

TABLE 11

| | | | | | | | | | FFA |
|---|---|---|---|---|---|---|---|---|---|
| | | | Glycerine | MeOH in | | Enzyme | | | (wt %) |
| | Flow | FFA | Dosage | Glycerine | Temp | (% wt/wt | FFA (wt %) | FFA (wt %) | Reactor |
| Comments | (kg/h) | (wt %) | (wt %) | (wt %) | (° C.) | oil) | Reactor 1 | Reactor 2 | 3 |
| Startup | 7000 | 9.52 | 13 | 22 | 43 | 0.17 | 1.55 | 0.94 | 1.29 |
| Increased | 10000 | 9.59 | 13 | 22 | 43 | 0.17 | 1.38 | 1.04 | 0.81 |
| flow | | | | | | | | | |
| Blending | 7000 | 12.65 | 13 | 16 | 43 | 0.1 | 3.43 | 1.64 | 1.24 |
| of | | | | | | | | | |
| feeds | | | | | | | | | |

The results show a snapshot of the most important variations tested during the trial. The product of R3 went through an evaporation step and a gravitational settling separation step before entering the alcoholic neutralization. Following evaporation and separation, FFA levels were largely below 1.5 wt %, although a few datapoints reached up to 1.9 wt %, which was due to a test using adding too much additional methanol. FFA below 1.5 wt % was acceptable concentrations for the alcoholic neutralization step at this specific industrial producer. After alcoholic neutralization, the product oil had appropriate quality and was able to enter the chemical transesterification with a good final biodiesel product quality. The biodiesel producer has now implemented the process and has procured the enzyme as a testimony to the viability of the process of the invention. Additionally, this change shows its competitive features which resulted in a substitution of the existing acid-catalyzed esterification process for the more benign process of the invention.

### Advantages of the Invention

The examples have shown that FFA can be reduced significantly using economically viable dosages of two relevant enzymes with different selectivity profiles. The most preferable employment of the invention is through use of a TAG-inactive enzyme, preferably, but not limited to, SEQ ID NO:1. Most preferably, an amount of added glycerol exceeding 5% is employed. Most preferably, an optimal amount of methanol is dosed quickly to enable fast and efficient reduction of FFA without inactivating the enzyme significantly. This optimal dosage and dosage rate will depend on the added amount of glycerol and/or water. Further, Temperature, pH, and water addition or even, conversely, extent of drying should be optimized too. 35-45° C. is optimal for SEQ ID NO: 1, however, depending on water and methanol concentration. pH 4.5-7 is typically a stable operating range for SEQ ID NO:1. Mixing should be as efficient as possible. Further, it is preferable to recycle the enzyme using the methods known in the art.

For an oil feedstock comprising 8% FFA, which results in around 0.5% water when fully esterified, the examples suggest that 2-4% methanol dosed near instantaneously, when 10% glycerol and 0.5% is present will yield a fine result. However, the results also show a robust application, with room for large variations while still achieving the target FFA levels of the invention.

All optimizations must be done especially according to the amount of FFA in the oil feedstock. Relevant feedstocks may comprise any amount of FFA ranging from just above 1% and up to 100%. For example, having a feedstock with 1% FFA, while having no alcoholic neutralization setup, and while requiring FFA<0.2 wt %, means reduction of FFA must happen through deodorization or neutralization today. In such cases the invention allows for reduction of FFA using the examples above, while for example using the methanol-containing heavy glycerol phase stemming from the base-catalyzed transesterification reaction after neutralization of the base by use of e.g. inorganic or organic acid.

Similarly, an oil feedstock exceeding 30% FFA may also be converted using the invention. Esterification of 100% FFA results in around 6% water, which, at equilibrium, will likely result in unacceptably high concentrations of FFA. Thus, conversion of feedstocks with such FFA levels would preferably include a means of drying or separation of water between a number of reaction steps.

Most preferably, the feedstock oil comprises between 2 and 30 wt % FFA. Being above 2 wt % makes neutralization (saponification) by NaOH or KOH expensive due to the accompanying yield loss of oil in the soap, while deodorization will be expensive in all cases due to the cost of procurement of the equipment. Below 2%, saponification and subsequent acidulation may be economically competitive and simpler, but the invention is still a viable option at least technically.

Converting feedstocks at around 30 wt % FFA results in water levels upon reaction that do not exceed 2-3%, above which achievable FFA concentrations at equilibrium may be unacceptable, unless the reaction mixture is somehow dried during the process to keep water levels low enough to arrive at acceptable FFA concentrations.

1. A process for reduction of free fatty acid concentrations in a fatty acid feedstock by enzymatic esterification with alcohol thereby reducing free fatty acid concentrations to levels compatible with a base-catalyzed biodiesel production process, or as a crude oil product suitable as fuel for ships, or as a feedstock oil for hydrotreated vegetable oil (HVO) production comprising steps of:
(a) providing fatty acid feedstock comprising more than 1 wt % Free Fatty Acids (FFA);
(b) contacting said fatty acid feedstock with a composition comprising an esterase and at least one component selected from the group consisting of alcohol, alcohol and glycerol, alcohol and water, alcohol and water and glycerol to form a mixture; and
(c) incubating the mixture of step (b) under conditions allowing the esterase to catalyze esterification of FFA until FFA levels are reduced either:
i) below 10 wt % if a neutralization step follows the enzymatic esterification reaction;
ii) below 2 wt % if a base-catalyzed transesterification follows the enzymatic esterification reaction without further FFA reduction; or
iii) below 10 wt %, below 5 wt %, such as below 2 wt % if neither neutralization nor base catalyzed transesterification follows the esterification step c).

2. The process of claim 1, wherein the product of step (c) (i) is separated into a first light phase L1 (6) comprising esterified oil and FFA below 10 wt %, and a first heavy phase H1 (7) comprising glycerol.

3. The process of claim 1, wherein the first light phase L1 (6) or the reaction mixture of step (c) (i) is neutralized (2), preferably by alcoholic neutralization, and with optional further addition of alcohol and/or base, resulting in an FFA level below 2 wt %.

4. The process of claim 1, wherein neutralization is followed by separation into a second light phase L2 (14) comprising neutralized oil and FFA, and a second heavy phase H2 (8) comprising neutralized glycerol.

5. The process of claim 4, wherein the neutralized oil in the second light phase L2 (14) is reacted in a base catalyzed transesterification reaction (3) in presence of alcohol resulting in a biodiesel reaction mixture comprising alkyl ester.

6. The process of claim 1, wherein the product of step (c) (ii) is separated into a first light phase L1 (6) comprising esterified oil and FFA below 2 wt %, and a first heavy phase H1 (7) comprising glycerol.

7. The process of claim 6, wherein the esterified oil in the first light phase L1 (6) is reacted in a base catalyzed transesterification reaction (3) in presence of alcohol resulting in a biodiesel reaction mixture comprising alkyl ester.

8. The process of claim 5, wherein the biodiesel reaction mixture comprising alkyl ester is separated into a third light phase L3 (18) comprising a crude biodiesel/alkyl ester product, and a third heavy phase H3 (13) comprising alkaline crude glycerol.

9. The process of claim 5, wherein the third heavy phase H3 (13) is recycled back to the neutralization step (2).

10. The process of claim 1, wherein the reaction mixture of step (c) (ii) is reacted in a base catalyzed transesterification reaction (3) in presence of alcohol resulting in a biodiesel reaction mixture comprising alkyl ester.

11. The process of claim 1, wherein the second heavy phase H2 (8) and/or third heavy phase H3 (13) is recycled back to step (b).

12. The process of claim 1, wherein the reaction time in step (c) is less than 48 hours.

13. The process according to claim 1 comprising the steps of:
(a) providing a fatty acid feedstock comprising free fatty acids (FFA) in the range from 2 wt % to 30 wt %;
(b) contacting said fatty acid feedstock with a composition comprising a TAG-inactive esterase and alcohol and glycerol; and
(c) incubating the mixture of step (b) under conditions allowing the esterase to catalyze esterification of FFA until FFA levels are reduced below 2 wt %; and further comprising
(d) separating the product of step (c) into a first light phase L1 and a first heavy phase H1; and
(e) neutralizing the residual FFA in the light phase L1 in an alcoholic neutralization step (2); and
(f) separation of the neutralization product of step (e) in a second light phase L2 (14) and a second heavy phase H2 (8); and
(g) base catalyzed transesterification of the light phase L2 (14) to produce a crude biodiesel reaction mixture.

14. The process according to claim 1 comprising the steps of:
(a) providing fatty acid feedstock comprising more than 1 wt % Free Fatty Acids (FFA);
(b) contacting said fatty acid feedstock with a composition comprising an esterase and alcohol and glycerol; and
(c) incubating the mixture of step (b) under conditions allowing the esterase to catalyze esterification of FFA until FFA levels are reduced below 1 wt %; and in case the amount of water in the incoming streams and FFA in the feedstock results in water concentrations that leads to FFA concentrations exceeding 1 wt % at equilibrium, then at least one of the following steps is applied:
(c) (i) Drying the reaction mixture, and/or
(c) (ii) Drying incoming streams of oil, and/or glycerol, and/or
(c) (iii) Separating the light phase from the heavy phase, drying the heavy phase, and recycling at least part of the dried heavy phase back into step (c);
(d) separating the product of step (c) into a first light phase L1 (6) and a first heavy phase H1 (7), which optionally is combined with step (c) (iii) above; and
(e) base catalyzed transesterification of the second light phase L2 to produce a crude biodiesel reaction mixture.

15. The process according to claim 1, wherein the esterase is selected from an esterase having at least 80% sequence identity to the polypeptide of SEQ ID NO: 1.

16. A process for producing a crude biodiesel mixture, said process comprising an enzymatic pretreatment according to claim 1, and further comprising at least one separation step of a light and a heavy phase, and comprising at least a base catalyzed transesterification step to produce the crude biodiesel mixture comprising alkyl ester.

17. The process of claim 1, wherein the alcohol is selected from methanol and ethanol.

18. An enzymatic process for producing an oil suitable as fuel for ships and/or as an oil feedstock for further downstream processing not involving transesterification, comprising the steps of:
(a) providing fatty acid feedstock comprising more than 1 wt % Free Fatty Acids (FFA);
(b) contacting said fatty acid feedstock with a composition comprising an esterase and at least one component selected from the group consisting of alcohol, alcohol and glycerol, alcohol and water, alcohol and water and glycerol to form a mixture; and
(c) incubating the mixture of step (b) under conditions allowing the esterase to catalyze esterification of FFA until FFA levels are reduced below 10 wt %, below 5 wt %, such as below 2 wt %.

19. The process of claim 18, wherein the alcohol is methanol or ethanol.

20. The process of claim 18, further comprising a hydrotreatment step, yielding aliphatic hydrocarbons.

21. The process according to claim 1, wherein the esterase is selected from an esterase having at least 80% sequence identity to the polypeptide of SEQ ID NO: 1, SEQ ID NO: 2, SEQ ID NO: 3, or SEQ ID NO: 4, particularly SEQ ID NO: 1.

22. The process according to claim 1, wherein the esterase is a TAG-inactive esterase, or is substantially free of TAG-activity, such as SEQ ID NO: 1.

23. The process of claim 22, wherein the esterase is not immobilized.

24. (canceled)


SEQUENCE LISTING (4 sequences)

SEQ ID NO: 1 - protein, 323 aa, Candida antarctica
TPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 2 - protein, 268 aa, Thermomyces lanuginosus
EVSQDLFNQFNLFAQYSAAAYCGKNNRAPAGTNITCTANACPEVEKADATFLYSFEDSGVGDVTGFLALDNTNKLIVLSFRGSRSIENWIGNLNFELKEINDICSGCRGHAGFTSSWRSVADTLRQKVEDAVREHPDYRVVFTGHSLGGALATVAGADLRGNKYDIDVFSYGAPRVGNRAFAEFLTVQTGGTLYRITHTNDIVPRLPPREFGYSHSSPEYWISGTLVPVRRRDIVKIEGIDATGGNNQPNIPSITAHLWYFGLIGTCL

SEQ ID NO: 3 - protein, 273 aa, Thermomyces lanuginosus
EAEACVSQDLFNQFNLFAQYSAAAYCGKNNDAPAGTNITCTANACPEVEKADATFLYSFEDSGVGDVTGFLALDNTNKLIVLSFRGSRSIENWIGNLNFELKEINDICSGCRGHAGFTSSWRSVADTLRQKVEDAVREHPDYRVVFTGHSLGGALATVAGADLRGNKYDIDVFSYGAPRVGNRAFAEFLTVQTGGTLYRITHTNDIVPRLPPREFGYSHSSPEYWIKSGTLVPVRRCDIVKIEGIDATGGNNQPNIPSITAHLWYFGLIGTCL

SEQ ID NO: 4 - protein, 319 aa, Chromobacterium viscosum
ADTYAATRYPVILVHGLAGTDKFANVVDYWYGIQSDLQSHGAKVYVANLSGFQSDDGPNGRGEQLLAYVKQVLAATGATKVNLIGHSQGGLTSRYVAAVAPQLVASVTTIGTPHRGSEFADFVQDVLKTDPTGLSSTVIAAFVNVFGTLVSSSHNTDQDALAALRTLTTAQTATYNRNFPSAGLGAPGSCQTGAATETVGGSQHLLYSWGGTAIQPTSTVLGVTGATDTSTGTLDVANVTDPSTLALLATGAVMINRASGQNDGLVSRCSSLFGQVISTSYHWNHLDEINQLLGVRGANAEDPVAVIRTHVNRLKLQGV